(19) United States
(12) Patent Application Publication
(10) Pub. No.: US 2025/0057882 A1
(43) Pub. Date: Feb. 20, 2025

(54) Transgenic Immune Cell, Construction Method Therefor and Use Thereof

(71) Applicant: FAPON BIOTHERAPY INC., Shenzhen, Guangdong (CN)

(72) Inventors: Xianjin WANG, Shenzhen, Guangdong (CN); Liang PENG, Shenzhen, Guangdong (CN); Lijun YE, Shenzhen, Guangdong (CN); Qian HUANG, Shenzhen, Guangdong (CN)

(21) Appl. No.: 18/721,462

(22) PCT Filed: Dec. 19, 2022
(86) PCT No.: PCT/CN2022/140094

§ 371 Date: Jun. 18, 2024

(30) Foreign Application Priority Data
Dec. 21, 2021 (CN) 202111572804.5

(51) Int. Cl. A61K 35/17; A61K 39/00; A61P 35/00; C07K 14/52; C07K 14/54; C07K 14/725; C07K 16/28; C07K 16/32; C07K 16/40; C12N 5/0783; C12N 15/86

(52) U.S. Cl. CPC A61K 35/17; A61K 39/4611; A61K 39/4631; A61K 39/4635; A61K 39/464454; A61K 39/464468; A61P 35/00; C07K 14/521; C07K 14/54; C07K 14/7051; C07K 16/2818; C07K 16/32; C07K 16/40; C12N 5/0636; C12N 15/86; A61K 2239/13; C07K 2317/565; C07K 2317/622; C07K 2319/03; C07K 2319/30; C12N 2740/15041

(57) ABSTRACT

The present application relates to the technical field of chimeric antigen receptor T cell immunotherapy (CART), and in particular to a transgenic immune cell, and a construction method therefor and a use thereof. The present application provides a gene containing three coding regions, a recombinant nucleic acid containing the gene, a biological material, and a transgenic immune effector cell, wherein three functional proteins can be coded, and the expression of the three proteins enables an immune effector cell to have multiple functions, thereby reducing the inhibition effect of a solid tumor microenvironment on the immune effector cell, prolonging the time of the killing effect of the immune effector cell, and improving the anti-tumor efficacy of the immune effector cell.

### CROSS-REFERENCE TO RELATED APPLICATION

The present application is a National Stage of International Patent Application No. PCT/CN2022/140094, filed on Dec. 19, 2022, and claims priority to Chinese patent application (Application No: 202111572804.5, and entitled: Transgenic Immune Cell, Construction Method Therefor and Use thereof) filed on Dec. 21, 2021, the disclosure of which is hereby incorporated by reference in its entirety.

### SEQUENCE LISTING

The instant application includes a Sequence Listing that has been submitted electronically in XML format and is hereby incorporated by reference in its entirety. The said XML file, named PN261085FPSW-seq.xml, is 39,714 bytes in size. The sequence listing includes 32 sequences with SEQ ID NOs: 1 to 32, which are substantially identical in substance to the sequences disclosed in the PCT application. The sequence listing does not include any new matter.

### TECHNICAL FIELD

The present disclosure relates to the technical field of chimeric antigen receptor T cell immunotherapy (CART), and in particular to a transgenic immune cell, a construction method therefor and use thereof.

### BACKGROUND

The chimeric antigen receptor T cell immunotherapy, CART for short, is a method of treating a patient by modifying the patient's T cells in vitro, enabling the patient's T cells to have the ability to recognize tumor cells, and then expanding the culture in vitro before being reintroduced into the patients' body. At present, CART using CD19 as a target achieves tremendous results in the treatment of B cell hematological tumors. However, according to clinical research results, the efficacy of CD19 CART in the treatment of B cell lymphoma is far inferior to that in the treatment of B cell acute lymphoblastic leukemia. This may be because the B cell lymphoma is a solid tumor, and it is difficult for CART cells to reach and infiltrate the tumor in large quantities. For the CART cells infiltrating the interior of the tumor, solid tumor cells may evade CART cell killing by overexpressing a large number of PD-L1 molecules on the surface. At the same time, the insufficient contact between the CART cells and the solid tumor cells also significantly affects CART cell proliferation and persistence, thus the rate of CART cell depletion in vivo is accelerated.

Therefore, compared with the treatment of hematological malignancy, the treatment of the solid tumors requires the higher requirements for the CART cells, and the CART cells need to have the ability to resist and even destroy the microenvironment of the solid tumors, and continue to penetrate the interior of the solid tumors to exert therapeutic effects.

Cytokines are a type of small molecular proteins with broad biological activity synthesized and secreted by immune cells (such as monocytes, macrophages, T cells, B cells, NK cells and the like) and certain non-immune cells (endothelial cells, epidermal cells, fibroblasts and the like) through stimulation, which regulate cell growth, differentiation, and effects by binding to corresponding receptors, as to regulate immune responses. Interleukins are one of the important classifications, and has various functions such as immune regulation, hematopoiesis, and inflammation regulation. At present, more than thirty types of the interleukins are reported already, among which IL-21 is generated by CD4 T cells and NK T cells, to stimulate the maturation of CD8 T cells and NK cells and enhance its cytotoxicity. It also has a function of promoting the differentiation of memory CD8 T cells and the like. The numerous efficacy of IL-21 makes it become a potential target for immunotherapy, but its extensive expression includes T cells, B cells, NK cells, and bone marrow cells.

Chemokines are a type of small cytokines or signal proteins secreted by cells. Since they have the ability to induce directional chemotaxis of nearby reactive cells, they are named as the chemokines. The chemokines may be divided into four subgroups, CXC, CC, C, and CX3C, according to the arrangement mode of its amino-terminus (N-terminus) cysteine, herein, CCL19 and CCL21 of the CC chemokine subgroup are ligands of CCR7, and the T cells express a CCR7 protein. Therefore, CCL19 and CCL21 chemokines may promote T cell migration.

The above cytokines and chemokines are all beneficial for resisting and improving the microenvironment of the solid tumors, and providing a long-lasting killing environment for the immune cells.

### SUMMARY

How to integrate a plurality of functional molecules on the basis of existing immune cells to jointly resist the microenvironment of solid tumors and significantly improve the therapeutic effect of immune cells is an urgent problem that needs to be solved at present.

A purpose of the present disclosure is to provide a multifunctional immune cell for the microenvironment of the solid tumors, with the expectation of not only resisting the microenvironment gradually formed during the development of the solid tumors, which is beneficial for its stability and spread, but also promoting autoimmune cells, maximizing the activation of the body's own immune system, and improving the strength of the body's fight against the solid tumors.

In order to solve the above problem and achieve the above purpose, the present disclosure provides the following technical schemes:

In a first aspect, the present disclosure provides a gene comprising three coding regions, herein, a coding region (I) encodes a chimeric antigen receptor, and the chimeric antigen receptor comprises an extracellular region that specifically recognizes a tumor antigen; a coding region (II) encodes a fusion protein, and the fusion protein comprises an immune checkpoint antibody and a cytokine; and a coding region (III) encodes a chemokine.

In an optional implementation mode, the tumor antigen is selected from at least one of MSLN, GD2, GPC3, CD19, EGFR VIII, GUCY2C, HER2, MUC16, or Claudin 18.2.

In an optional implementation mode, the extracellular region comprises an anti-MSLN antibody.

In an optional implementation mode, the extracellular region comprises an anti-GUCY2C antibody; and in an optional implementation mode, the chimeric antigen receptor comprises an anti-GUCY2C single chain antibody.

Preferably, the amino acid sequence of the anti-MSLN antibody is (a) an amino acid sequence as shown in SEQ ID NO: 1, or (b) a derivative amino acid sequence of SEQ ID NO: 1 that is substituted, deleted, or added with one or more amino acids in the amino acid sequence defined in (a) and encodes a protein with specific recognition of MSLN antigen activity.

In an optional implementation mode, the immune checkpoint is selected from at least one of PD1, PD-L1, TIGIT, LAG3, CTLA4, BTLA or TIM3.

In an optional implementation mode, the immune checkpoint antibody is an anti-PD1 antibody.

Preferably, the amino acid sequence of the anti-PD1 antibody is (c) an amino acid sequence as shown in SEQ ID NO: 2, or (d) a derivative amino acid sequence of SEQ ID NO: 2 that is substituted, deleted, or added with one or more amino acids in the amino acid sequence defined in (c) and encodes a protein with a PD1 targeting function.

In an optional implementation mode, the cytokine is (A) or (B);

(A) is selected from at least one of IL-21, IL-23, IL-2, IL-7, IL-9, IL-12, IL-15, or IL-18; and

(B) is a protein derived from (A) that has immune cell regulating activity.

In an optional implementation mode, the cytokine is IL-21.

Preferably, IL-21 has an amino acid sequence as shown in SEQ ID NO: 3.

In an optional implementation mode, the chemokine is (c) or (d);

(c) is selected from at least one of CXC chemokine, CC chemokine, CX3C chemokine, or XC chemokine; and

(d) is a protein derived from (c) that has a function of inducing directed migration of the immune cell.

Preferably, the CXC chemokine is selected from at least one of CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 or CXCL17.

Preferably, the CC chemokine is selected from at least one of CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27 or CCL28.

Preferably, the CX3C chemokine is CX3CL1.

Preferably, the XC chemokine is XCL1.

In an optional implementation mode, the chemokine is CCL19 or CCL21.

Preferably, CCL19 has an amino acid sequence as shown in SEQ ID NO: 4.

Preferably, CCL21 has an amino acid sequence as shown in SEQ ID NO: 5.

In a second aspect, the present disclosure provides a recombinant nucleic acid, the recombinant nucleic acid comprises a first nucleic acid molecule, a second nucleic acid molecule, and a third nucleic acid molecule, the first nucleic acid molecule comprises the coding region (I) according to any one of the aforementioned implementation modes, the second nucleic acid molecule comprises the coding region (II) according to any one of the aforementioned implementation modes, and the third nucleic acid molecule comprises the coding region (III) according to any one of the aforementioned implementation modes.

In an optional implementation mode, the first nucleic acid molecule, the second nucleic acid molecule, and the third nucleic acid molecule are linked by a nucleic acid sequence of a 2A peptide.

Preferably, the 2A peptide is selected from at least one of P2A, T2A, F2A, or E2A.

In an optional implementation mode, the first nucleic acid molecule is linked to the second nucleic acid molecule by the nucleic acid sequence of the 2A peptide, and the second nucleic acid molecule is linked to the third nucleic acid molecule by the nucleic acid sequence of the 2A peptide.

In a third aspect, the present disclosure provides a biomaterial, and the biomaterial comprises any one of the following:
(i) a recombinant vector, herein the recombinant vector comprises the gene according to any one of the aforementioned implementation modes, or the recombinant nucleic acid according to any one of the aforementioned implementation modes; and
(ii) a construct, herein the construct comprises a non-pathogenic virus, and the non-pathogenic virus comprises the gene according to any one of the aforementioned implementation modes, the recombinant nucleic acid, or the recombinant vector in (i).

In an optional implementation mode, the non-pathogenic virus comprises a retrovirus, a lentivirus, or an adenovirus.

In a fourth aspect, the present disclosure provides a transgenic immune effector cell, and the transgenic immune effector cell comprises the gene, the recombinant nucleic acid or the biomaterial according to any one of the aforementioned implementation modes.

In an optional implementation mode, a construction method for the transgenic immune effector cell comprises introducing the gene, the recombinant nucleic acid or the biomaterial according to any one of the aforementioned implementation modes into an immune effector cell, to obtain the transgenic immune effector cell.

The immune effector cell is selected from at least one of T cell, NK cell, NKT cell, macrophage, or CIK cell.

Preferably, the immune effector cell is the T cell.

In a fifth aspect, the present disclosure provides an application of the gene, the recombinant nucleic acid, the biomaterial, the transgenic immune effector cell, the transgenic immune effector cell constructed by the construction method according to any one of the aforementioned implementation modes in preparation of an anti-tumor product.

Preferably, the tumor comprises a solid tumor.

In a sixth aspect, the present disclosure provides an anti-tumor drug, and the drug comprises at least one of the gene, the recombinant nucleic acid, the biomaterial, or the transgenic immune effector cell according to any one of the aforementioned implementation modes.

Preferably, the tumor comprises a solid tumor.

In a seventh aspect, the present disclosure provides a method for treating a tumor, and the method comprises administering a therapeutically effective amount of at least one of the gene, the recombinant nucleic acid, the biomaterial, or the transgenic immune effector cell to a subject according to any one of the aforementioned implementation modes. Preferably, the tumor comprises a solid tumor.

In an eighth aspect, the present disclosure provides a pharmaceutical composition, and it comprises at least one of the gene, the recombinant nucleic acid, the biomaterial, or the transgenic immune effector cell according to any one of the aforementioned implementation modes, as well as a pharmaceutically acceptable carrier.

The gene, the recombinant nucleic acid, the biomaterial, and the transgenic immune effector cell provided by the present disclosure may all encode three proteins. The expression of the three proteins enables the immune effector cell to have a plurality of functions simultaneously, comprising specifically recognizing the tumor antigen and targeting the immune checkpoint, as well as promoting and regulating the immune cell activity, thereby the inhibitory effect of the solid tumor microenvironment on the immune effector cell is reduced, and the killing time of the immune effector cell is prolonged; at the same time, the expression of the chemokine may recruit and promote the T cells of the body itself to reach the tumor site, and under the regulation of the cytokine, the tumor cells are killed jointly, thus the anti-tumor efficacy of the immune effector cell is enhanced.

### BRIEF DESCRIPTION OF THE DRAWINGS

In order to describe specific implementation modes of the present disclosure or technical schemes in existing technologies more clearly, drawings required in the description of the specific implementation modes or the existing technologies are briefly introduced below. Apparently, the drawings in the following description are some implementation modes of the present disclosure. For those skilled in the art, other drawings may also be obtained according to these drawings without paying creative labor.

FIG. 1 is a composition schema graph of recombinant nucleic acid sequences used in Embodiment 1, Embodiment 2, and Comparative example 1 of the present disclosure (namely, a schema graph of a lentiviral vector coding gene);

FIG. 2 is a positivity detection result of CART cells provided in Embodiment 3, Embodiment 4, and Comparative example 1 of the present disclosure;

FIG. 3 is a comparative result of in vitro tumor killing ability of the CART cells provided in Embodiment 3, Embodiment 4, and Comparative example 1 of the present disclosure;

FIG. 4 is a comparison result of chemotaxis ability of the CART cells provided in Embodiment 3, Embodiment 4, and Comparative example 1 of the present disclosure;

FIG. 5 is a composition schema graph of recombinant nucleic acids used in Embodiment 5 and Comparative example 2 of the present disclosure (namely, the schema graph of the lentiviral vector coding gene);

FIG. 6 is a positivity detection result diagram of CART cells in Embodiment 5 of the present disclosure;

FIG. 7 is a comparative result diagram of in vitro tumor killing ability of CART cells in Embodiment 6 of the present disclosure; and

FIG. 8 is a result diagram of an in vivo tumor inhibition experiment in Embodiment 7 of the present disclosure.

### DETAILED DESCRIPTION OF THE EMBODIMENTS

In order to make purposes, technical schemes, and advantages of embodiments of the present disclosure clearer, the technical schemes in the embodiments of the present disclosure are clearly and completely described below in combination with drawings in the embodiments of the present disclosure. Apparently, the embodiments described are a part of the embodiments of the present disclosure, not all of the embodiments. Usually, components of the embodiments of the present disclosure described and shown in the drawings here may be arranged and designed in various different configurations.

Therefore, the following detailed description of the embodiments of the present disclosure provided in the drawings is not intended to limit the scope of the present disclosure claimed to be protected, but only to represent the selected embodiments of the present disclosure. Based on the embodiments in the present disclosure, all other embodiments obtained by those of ordinary skill in the art without paying creative labor shall fall within the scope of protection of the present disclosure.

It should be noted that: similar labels and letters represent similar terms in the following drawings. Therefore, once a certain term is defined in one drawing, it does not need to be further defined or explained in the subsequent drawings.

In addition, terms “first” and “second” are only used for a descriptive purpose, and may not be understood as indicating or implying relative importance or implicitly indicating the number of technical features indicated. Thus, the features defined as the “first” and “second” may explicitly or implicitly comprise at least one such feature. In the description of the present disclosure, “multiple” means at least two, such as two, and three, unless otherwise specifically defined.

In a certain specific implementation mode, in a first aspect, the present disclosure provides a gene comprising three coding regions, herein, a coding region (I) encodes a chimeric antigen receptor, and the chimeric antigen receptor comprises an extracellular region that specifically recognizes a tumor antigen;
a coding region (II) encodes a fusion protein, and the fusion protein comprises an immune checkpoint antibody and a cytokine; and
a coding region (III) encodes a chemokine.

The gene of the present disclosure should be understood as all genes comprising the three coding regions mentioned above. As for whether there are other coding regions besides the three coding regions mentioned above, it is not limited in the present disclosure. Those skilled in the art may choose to add other coding regions according to actual needs.

The above three coding regions of the present disclosure are aimed at independent expression, therefore, the linkage order of the three coding regions in the above gene of the present disclosure may be selected according to actual needs without special limitations.

The gene of the present disclosure may also comprise an appropriate intron in the case without affecting the independent expression of the three coding regions.

The above chimeric antigen receptor refers to a chimeric protein that is formed by coupling a chimeric antigen receptor T cell with a transmembrane region and an intracellular region in vitro through an antigen binding site of an antibody recognizing a certain tumor antigen. It is then transfected into a T cell of a patient by a gene transduction method, so that it expresses the chimeric antigen receptor, and the T cell of the patient may generate a large number of tumor specific CART cells after being “recoded”. After the recoded chimeric antigen receptor T cells are added to the patient's body, the chimeric antigen receptor may specifically track, recognize, and guide the T cells to kill tumor cells. The present disclosure extends the application scope of the chimeric antigen receptor to other immune effector cells, such as T cell, NK cell, NKT cell, macrophage, or CIK cells, based on existing CART cells. The antigen binding site of the antibody is an extracellular region recognizing the tumor antigen as described in the present disclosure, while the transmembrane region and the intracellular region may be obtained by conventional selections according to actual needs.

Preferably, the transmembrane region is a transmembrane segment of CD8.

Preferably, the intracellular region is an intracellular segment of an immune co-stimulatory molecule and a CD3 Zeta chain.

Preferably, the immune co-stimulatory molecule is selected from any one or more of 4-1BB, CD28, CD3, OX-40, CD40L, CD27, CD30, or their derivatives. In an optional implementation mode, the immune co-stimulatory molecule is selected from 4-1BB.

In an optional implementation mode, the tumor antigen of the present disclosure is selected from at least one of MSLN, GD2, GPC3, CD19, EGFR VIII, GUCY2C, HER2, MUC16, or Claudin 18.2.

In an optional implementation mode, the extracellular region comprises an anti-MSLN antibody.

Preferably, the amino acid sequence of the anti-MSLN antibody is (a) an amino acid sequence as shown in SEQ ID NO: 1, or (b) a derivative amino acid sequence of SEQ ID NO: 1 that is substituted, deleted, or added with one or more amino acids in the amino acid sequence defined in (a) and encodes a protein with specific recognition of MSLN antigen activity.

Amino acid sequence of anti-MSLN antibody (SEQ ID NO: 1):

QVQLQQSGPELEKPGASVKLSCKASGYSFTGYTMNWVKQSHGKSL
EWIGLITPYNGASSYNQKFRGKATLTVDKSSSTAYMDLLSLTSED
SAVYFCARGGYDGRGFDYWGQGTTVTVSSGGGGSGGGGSGGGGSD
IELTQSPAIMSASPGEKVTMTCSASSSVSYMHWYQQKSGTSPKRW
IYDTSKLASGVPGRFSGSGSGNSYSLTISSVEAEDDATYYCQQWS
KHPLTFGAGTKLEIK.

The term “antibody” is used in the broadest sense, and it encompasses various antibody structures, comprising but not limited to monoclonal antibodies/polyclonal antibodies, multispecific antibodies (such as bispecific antibodies and trispecific antibodies), mouse-derived antibodies/chimeric antibodies, full-length antibodies or its antigen binding fragments (such as scFv), as long as they show the desired antigen binding activity.

The term “complementary determining region”, “CDR” or “CDRs” or “complementarity determining region” refers to a highly variable region of a heavy chain and a light chain of an immunoglobulin, and it is a region within a variable domain of the antibody that mainly promotes the specific binding with the antigen. The heavy chain complementary determining region is represented by HCDR, and the heavy chain variable region comprises three CDR regions: HCDR1, HCDR2, and HCDR3; and the light chain complementary determining region is represented by LCDR, and the light chain variable region comprises three CDR regions: LCDR1, LCDR2, and LCDR3. The amino acid sequence boundaries of CDR may be determined by various well-known schemes, for example: “Kabat” numbering rules (referring to Kabat et al. (1991), “Sequences of Proteins of Immunological Interest”, 5th edition, Public Health Service, National Institutes of Health, Bethesda, MD), “Chothia” numbering rules, “ABM” numbering rules, “contact” numbering rules (referring to Martin, ACR. Protein Sequence and Structure Analysis of Antibody Variable Domains [J]. 2001) and ImmunoGenTics (IMGT) numbering rules (Lefranc, M. P. et al., Dev. Comp. Immunol., 27, 55-77 (2003)); and the correspondence relationship between various numbering systems is well-known to those skilled in the art.

In an optional implementation mode, the chimeric antigen receptor comprises an anti-MSLN antibody. In an optional implementation mode, the anti-MSLN antibody is a single chain antibody (scFv, which is formed by directly linking a light chain variable region (VL) and a heavy chain variable region (VH) of the antibody or linking by a peptide linker (L). For example, an N-terminus to a C-terminus: scFv of VH-L-VL or VL-L-VH, the linker L may be selected from a (GxS) y linker, herein x is selected from an integer of 1-5 and y is selected from an integer of 0-6, for example, x is 4 and y is 3). In an optional implementation mode, the anti-MSLN antibody comprises a heavy chain variable region and a light chain variable region; the heavy chain variable region of the anti-MSLN antibody comprises HCDR1, HCDR2, and HCDR3 of the heavy chain variable region (SEQ ID NO: 9) in SEQ ID NO: 1, and the light chain variable region of the anti-MSLN antibody comprises LCDR1, LCDR2, and LCDR3 of the light chain variable region (SEQ ID NO: 10) in SEQ ID NO: 1; in some implementation modes, HCDR1, HCDR2, and HCDR3, as well as LCDR1, LCDR2, and LCDR3, are defined by an IMGT numbering system, a Kabat numbering system, a Chothia numbering system, a Contact numbering system, or an AbM numbering system. In some implementation modes, HCDR1, HCDR2, and HCDR3, as well as LCDR1, LCDR2, and LCDR3, are defined by the Kabat numbering system. In an optional implementation mode, the amino acid sequences of CDRs (defined by the Kabat numbering system) of the anti-MSLN antibody are as follows: HCDR1: GYTMN (SEQ ID NO: 11); HCDR2: LITPYNGASSYNQKFRG (SEQ ID NO: 12); HCDR3: GGYDGRGFDY (SEQ ID NO: 13); LCDR1: SASSSVSYMH (SEQ ID NO: 14); LCDR2: DTSKLAS (SEQ ID NO: 15); and LCDR3: QQWSKHPLT (SEQ ID NO: 16). In some implementation modes, the heavy chain variable region of the anti-MSLN antibody comprises the heavy chain variable region in SEQ ID NO: 1, and the light chain variable region of the anti-MSLN antibody comprises the light chain variable region in SEQ ID NO: 1. In some implementation modes, the anti-MSLN antibody comprises a heavy chain variable region and a light chain variable region, herein the heavy chain variable region comprises an amino acid sequence with at least 85% sequence identity with SEQ ID NO: 9, and the light chain variable region comprises an amino acid sequence with at least 85% sequence identity with SEQ ID NO: 10; in some implementation modes, the heavy chain variable region and the light chain variable region comprise HCDR1, HCDR2, HCDR3, LCDR1, LCDR2, and LCDR3 of any one of the above anti-MSLN antibodies; and in some implementation modes, the anti-MSLN antibody comprises a heavy chain variable region as shown in SEQ ID NO: 9 and a light chain variable region as shown in SEQ ID NO: 10. In some embodiments, the anti MSLN antibody is a single chain antibody, which comprises SEQ ID NO: 1. In some implementation modes, the anti-MSLN antibody is a single chain antibody, and it comprises SEQ ID NO: 1. In some implementation modes, the anti-MSLN antibody is a single chain antibody, it comprises a derivative amino acid sequence of SEQ ID NO: 1, the derivative amino acid sequence of SEQ ID NO: 1 is obtained by substituting, deleting, or adding one or more amino acids, and a protein edited by it has a MSLN targeting function.

In an optional implementation mode, the amino acid sequence of the heavy chain variable region of the anti-MSLN antibody (SEQ ID NO: 9):

QVQLQQSGPELEKPGASVKLSCKASGYSFTGYTMNWVKQSHGKSL
EWIGLITPYNGASSYNQKFRGKATLTVDKSSSTAYMDLLSLTSED
SAVYFCARGGYDGRGFDYWGQGTTVTVSS.

The amino acid sequence of the light chain variable region of the anti-MSLN antibody (SEQ ID NO: 10):

DIELTQSPAIMSASPGEKVTMTCSASSSVSYMHWYQQKSGTSPKR
WIYDTSKLASGVPGRFSGSGSGNSYSLTISSVEAEDDATYYCQQW
SKHPLTFGAGTKLEIK.

In an optional implementation mode, the tumor antigen of the present disclosure is selected from GUCY2C. In an optional implementation mode, the chimeric antigen receptor comprises an anti-GUCY2C antibody. In an optional implementation mode, the anti-GUCY2C antibody is a single chain antibody (scFv). In an optional implementation mode, the anti-GUCY2C antibody comprises a heavy chain variable region and a light chain variable region. In some implementation modes, the heavy chain variable region of the anti-GUCY2C antibody comprises HCDR1, HCDR2, and HCDR3 in SEQ ID NO: 17, and the light chain variable region of the anti-GUCY2C antibody comprises LCDR1, LCDR2, and LCDR3 in SEQ ID NO: 18; and in some implementation modes, HCDR1, HCDR2, and HCDR3, as well as LCDR1, LCDR2, and LCDR3, are defined by the IMGT numbering system, the Kabat numbering system, the Chothia numbering system, the Contact numbering system, or the AbM numbering system. In an optional implementation mode, the amino acid sequences of CDRs (defined by the IMGT numbering system) of the anti-GUCY2C antibody are as follows: HCDR1: GYTFTEYT (SEQ ID NO: 20); HCDR2: INPNNGGA (SEQ ID NO: 21); HCDR3: ARAPYYYGSSYYAMDY (SEQ ID NO: 22); LCDR1: ESVDNYGISF (SEQ ID NO: 23); LCDR2: AAS; LCDR3: QQSKEVPFT (SEQ ID NO: 24). In some implementation modes, the anti-GUCY2C antibody comprises a heavy chain variable region and a light chain variable region, herein the heavy chain variable region comprises an amino acid sequence with at least 85% sequence identity with SEQ ID NO: 17, and the light chain variable region comprises an amino acid sequence with at least 85% sequence identity with SEQ ID NO: 18; in some implementation modes, the heavy chain variable region and the light chain variable region comprise HCDR1, HCDR2, HCDR3, LCDR1, LCDR2, and LCDR3 of any one of the aforementioned anti-GUCY2C antibodies. In some implementation modes, the heavy chain variable region of the anti-GUCY2C antibody comprises SEQ ID NO: 17, and the light chain variable region of the anti-GUCY2C antibody comprises SEQ ID NO: 18. In some implementation modes, the anti-GUCY2C antibody comprises the heavy chain variable region as shown in SEQ ID NO: 17 and the light chain variable region as shown in SEQ ID NO: 18. In some implementation modes, the anti-GUCY2C antibody is a single chain antibody, and it comprises an amino acid sequence with at least 85% sequence identity with SEQ ID NO: 19; and in some implementation modes, the antibody comprises a heavy chain variable region and a light chain variable region of any one of the aforementioned anti-GUCY2C antibodies. In some implementation modes, the anti-GUCY2C single chain antibody comprises SEQ ID NO: 19.

The amino acid sequence of the heavy chain variable region of the anti-GUCY2C antibody (SEQ ID NO: 17):

EVQLQQSGPELVKPGASVKISCKTSGYTFTEYTMHWVKQSHGKSL
EWIGGINPNNGNTNYNQKFKGKATLTVDKSSSTAYMELRSLTSED
SAVYYCGRSPFVHYYDYYAMDYWGQGTSVTVSS

The amino acid sequence of the light chain variable region of the anti-GUCY2C antibody (SEQ ID NO: 18):

DIVLTQSPASLAVSLGQRATISCRASESVYNSGISFMNWFQQKPG
QPPKLLIYAASNQGSGVPARFSGSGSGTDFSLNIHPMEEDDTAMY
FCQQSKEVPFTFGSGTNLEIK.

The amino acid sequence of the anti-GUCY2C single chain antibody (SEQ ID NO: 19):

EVQLQQSGPELVKPGASVKISCKTSGYTFTEYTMHWVKQSHGKSL
EWIGGINPNNGNTNYNQKFKGKATLTVDKSSSTAYMELRSLTSED
SAVYYCGRSPFVHYYDYYAMDYWGQGTSVTVSSGGGGSGGGGSGG
GGSDIVLTQSPASLAVSLGQRATISCRASESVYNSGISFMNWFQQ
KPGQPPKLLIYAASNQGSGVPARFSGSGSGTDFSLNIHPMEEDDT
AMYFCQQSKEVPFTFGSGTNLEIK.

The immune checkpoint (immune checkpoint molecule) is an inhibitory pathway in an immune system, which is regulated by ligand/receptor interaction. It plays an important role in maintaining self immune tolerance, regulating the duration and amplitude of physiological immune responses, thereby the damage and destruction of normal tissues caused by the immune system are avoided. In the microenvironment of the solid tumor, tumor cells bind to an immune checkpoint receptor (such as a PD1 receptor) expressed by immune cells (such as a T cell) by overexpressing an immune checkpoint ligand (such as a PD-L1 ligand), the immune cell activity is inhibited, thereby it is avoided from being killed by the immune cells. In order to prevent the binding between the immune checkpoint ligands and receptors expressed by the immune cells and tumor cells, the present disclosure provides the above immune checkpoint antibody in a fusion protein, this antibody binds to the immune checkpoint expressed by the immune cells in advance, thereby the immune checkpoint ligand expressed by the tumor cells is shielded, so that the immune cells maintain the sustained killing activity.

In an optional implementation mode, the immune checkpoint of the fusion protein of the present disclosure is selected from at least one of PD1, PD-L1, TIGIT, LAG3, CTLA4, BTLA, or TIM3. In an optional implementation mode, the immune checkpoint of the fusion protein of the present disclosure is selected from PD1.

In an optional implementation mode, the immune checkpoint antibody is an anti-PD1 antibody.

Preferably, the amino acid sequence of the anti-PD1 antibody is (c) an amino acid sequence as shown in SEQ ID NO: 2, or (d) a derivative amino acid sequence of SEQ ID NO: 2 that is substituted, deleted, or added with one or more amino acids in the amino acid sequence defined in (c) and edits a protein with a PD1 targeting function.

The amino acid sequence of the anti-PD1 antibody (SEQ ID NO: 2):

MGWSCIILFLVATATGVHSEIVLTQSPATLSLSPGERATLSCRAS
QSVSSYLAWYQQKPGQAPRLLIYDASNRATGIPARFSGSGSGTDF
TLTISSLEPEDFAVYYCQQSSNWPRTFGQGTKVEIKRGGGGSGGG
GSGGGGSQVQLVESGGGVVQPGRSLRLDCKASGITFSNSGMHWVR
QAPGKGLEWVAVIWYDGSKRYYADSVKGRFTISRDNSKNTLFLQM
NSLRAEDTAVYYCATNDDYWGQGTLVTVSS.

In an optional implementation mode, the anti-PD1 antibody is a single chain antibody (scFv). In an optional implementation mode, the anti-PD1 antibody comprises a heavy chain variable region and a light chain variable region; in some implementation modes, the heavy chain variable region of the anti-PD1 antibody comprises HCDR1, HCDR2, and HCDR3 of the heavy chain variable region (SEQ ID NO: 25) in SEQ ID NO: 2, and the light chain variable region of the anti-PD1 antibody comprises LCDR1, LCDR2, and LCDR3 of the light chain variable region (SEQ ID NO: 26) in SEQ ID NO: 2; and in some implementation modes, HCDR1, HCDR2, and HCDR3, as well as LCDR1, LCDR2, and LCDR3, are defined by the IMGT numbering system, the Kabat numbering system, the Chothia numbering system, the Contact numbering system, or the AbM numbering system. In an optional implementation mode, the amino acid sequences of CDRs (defined by the Kabat numbering system) of the anti-PD1 antibody are as follows: HCDR1: NSGMH (SEQ ID NO: 27); HCDR2: VIWYDGSKRYYADSVKG (SEQ ID NO: 28); HCDR3: NDDY (SEQ ID NO: 29); LCDR1: RASQSVSSYLA (SEQ ID NO: 30); LCDR2: DASNRAT (SEQ ID NO: 31); and LCDR3: QQSSNWPRT (SEQ ID NO: 32). In some implementation modes, the heavy chain variable region of the anti-PD1 antibody comprises the heavy chain variable region in SEQ ID NO: 2, and the light chain variable region of the anti-PD1 antibody comprises the light chain variable region in SEQ ID NO: 2. In some implementation modes, the anti-PD1 antibody comprises a heavy chain variable region and a light chain variable region, herein the heavy chain variable region comprises an amino acid sequence with at least 85% sequence identity with SEQ ID NO: 25, and the light chain variable region comprises an amino acid sequence with at least 85% sequence identity with SEQ ID NO: 26; in some implementation modes, the heavy chain variable region and the light chain variable region comprise HCDR1, HCDR2, HCDR3, LCDR1, LCDR2, and LCDR3 of any one of the aforementioned anti-PD1 antibodies. In some implementation modes, the heavy chain variable region of the anti-PD1 antibody comprises the amino acid sequence of SEQ ID NO: 25, and the light chain variable region comprises the amino acid sequence of SEQ ID NO: 26. In some implementation modes, the anti-PD1 antibody comprises the heavy chain variable region as shown in SEQ ID NO: 25 and the light chain variable region as shown in SEQ ID NO: 26. In some implementation modes, the anti-PD1 antibody is a single chain antibody, and it comprises SEQ ID NO: 2. In some implementation modes, the anti-PD1 antibody is a single chain antibody, it comprises a derivative amino acid sequence of SEQ ID NO: 2, the derivative amino acid sequence of SEQ ID NO: 2 is obtained by substituting, deleting, or adding one or more amino acids, and a protein edited by it has a PD1 targeting function.

In an optional implementation mode, the amino acid sequence of the heavy chain variable region of the anti-PD1 antibody (SEQ ID NO: 25):

QVQLVESGGGVVQPGRSLRLDCKASGITFSNSGMHWVRQAPGKGL
EWVAVIWYDGSKRYYADSVKGRFTISRDNSKNTLFLQMNSLRAED
TAVYYCATNDDYWGQGTLVTVSS.

The amino acid sequence of the light chain variable region of the anti-PD1 antibody (SEQ ID NO: 26):

MGWSCIILFLVATATGVHSEIVLTQSPATLSLSPGERATLSCRAS
QSVSSYLAWYQQKPGQAPRLLIYDASNRATGIPARFSGSGSGTDF
TLTISSLEPEDFAVYYCQQSSNWPRTFGQGTKVEIK;

the above cytokines are low molecular weight soluble proteins induced by immunogens, mitogens, or other stimuli in various cells, which has various functions such as regulating of innate and adaptive immunity, blood cell generation, cell growth, and repairing of damaged tissues. According to the different functions, the cytokines may be divided into an interleukin, an interferon, a tumor necrosis factor superfamily, a colony stimulating factor, and a growth factor and the like. The numerous cytokines exert effects in the body by paracrine, autocrine, or endocrine or other modes, and have a plurality of physiological characteristics such as pleiotropy, overlap, antagonism, and synergy, and a very complex cytokine regulatory network is formed, to participate in various important physiological functions of the human body. There are functional and degree differences in the regulation of the different immune cells by the different cytokines. The cytokine in the fusion protein provided by the present disclosure may be selected according to the selected immune effector cell. For example, the interleukin may activate a T lymphocyte to generate an active mediator. Therefore, when the T cell is transgenically modified, the interleukin may be selected as the cytokine in the fusion protein.

In an optional embodiments, the cytokine of the present disclosure is (A) or (B);

(A) is selected from at least one of IL-21, IL-23, IL-2, IL-7, IL-9, IL-12, IL-15, or IL-18; and

(B) is a protein derived from (A) that has immune cell regulating activity.

In an optional implementation mode, the cytokine is IL-21.

Preferably, IL-21 has an amino acid sequence as shown in SEQ ID NO: 3.

The amino acid sequence of IL-21 (SEQ ID NO: 3):

(SEQ ID NO: 3)
MHKSSSQGQDRHMIRMRQLIDIVDQLKNYVNDLVPEFLPAPEDVE
TNCEWSAFSCFQKAQLKSANTGNNERIINVSIKKLKRKPPSTNAG
RRQKHRLTCPSCDSYEKKPPKEFLERFKSLLQKMIHQHLSSRTHG
SEDS.

The main function of the above chemokines is to manage the migration (homing) of white blood cells to their respective positions in inflammation and homeostasis processes. It specifically comprises (1) basic homing chemokine: a basic steady-state chemokine generated in thymus and lymphoid tissues, for example, chemokines CCL19 and CCL21 (expressed in lymph nodes and lymphatic endothelial cells) and its receptor CCR7 plays a steady-state function in cell homing; and (2) inflammatory homing chemokine: the inflammatory chemokine generates a high concentration during infection or injury, and determines the migration of inflammatory white blood cells to a damaged area. The typical inflammatory chemokine comprises CCL2, CCL3, CCL5, CXCL1, CXCL2 and CXCL8.

In an optional implementation mode, the chemokine of the present disclosure is (c) or (d);
(c) is selected from at least one of CXC chemokine, CC chemokine, CX3C chemokine, or XC chemokine; and
(d) is a protein derived from (c) that has a function of inducing directed migration of the immune cell.

Preferably, the CXC chemokine is selected from at least one of CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 or CXCL17.

Preferably, the CC chemokine is selected from at least one of CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27 or CCL28.

Preferably, the CX3C chemokine is CX3CL1.

Preferably, the XC chemokine is XCL1.

In an optional implementation mode, the chemokine is CCL19 or CCL21.

Preferably, CCL19 has an amino acid sequence as shown in SEQ ID NO: 4.

The amino acid sequence of CCL19 (SEQ ID NO: 4):

(SEQ ID NO: 4)
MALLLALSLLVLWTSPAPTLSGTNDAEDCCLSVTQKPIPGYIVRN
FHYLLIKDGCRVPAVVFTTLRGRQLCAPPDQPWVERIIQRLQRTS
AKMKRRSS.

Preferably, CCL21 has an amino acid sequence as shown in SEQ ID NO: 5.

The amino acid sequence of CCL21 (SEQ ID NO: 5):

(SEQ ID NO: 5)
MAQSLALSLLILVLAFGIPRTQGSDGGAQDCCLKYSQRKIPAKVV
RSYRKQEPSLGCSIPAILFLPRKRSQAELCADPKELWVQQLMQHL
DKTPSPQKPAQGCRKDRGASKTGKKGKGSKGCKRTERSQTPKGP.

In a second aspect, the present disclosure provides a recombinant nucleic acid, the recombinant nucleic acid comprises a first nucleic acid molecule, a second nucleic acid molecule, and a third nucleic acid molecule, the first nucleic acid molecule comprises the coding region (I) according to any one of the aforementioned implementation modes, the second nucleic acid molecule comprises the coding region (II) according to any one of the aforementioned implementation modes, and the third nucleic acid molecule comprises the coding region (III) according to any one of the aforementioned implementation modes.

In an optional implementation mode, the first nucleic acid molecule, the second nucleic acid molecule, and the third nucleic acid molecule are linked by a nucleic acid sequence of a 2A peptide.

Preferably, the 2A peptide is selected from at least one of P2A, T2A, F2A, or E2A.

In an optional implementation mode, the first nucleic acid molecule is linked to the second nucleic acid molecule by the nucleic acid sequence of the 2A peptide, and the second nucleic acid molecule is linked to the third nucleic acid molecule by the nucleic acid sequence of the 2A peptide.

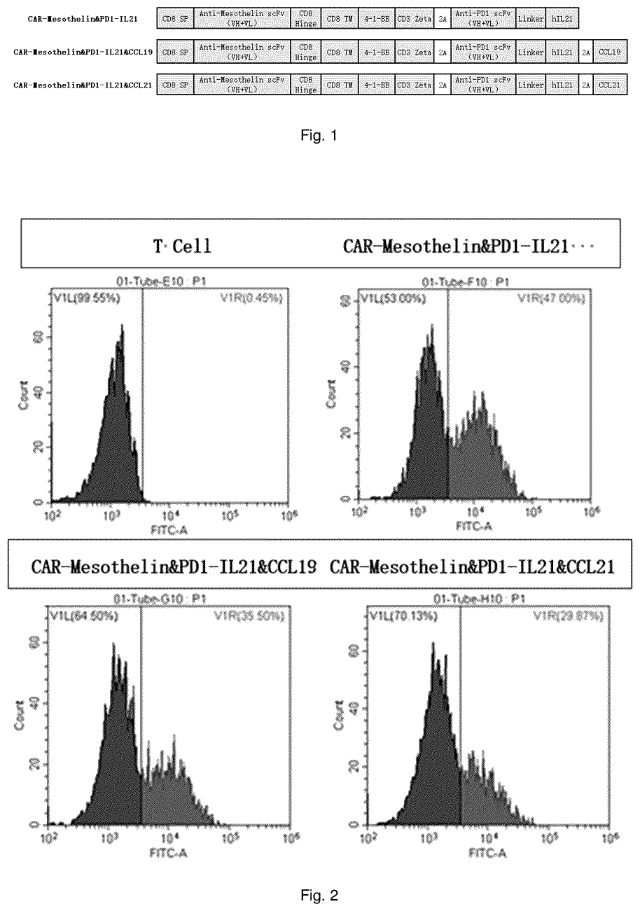

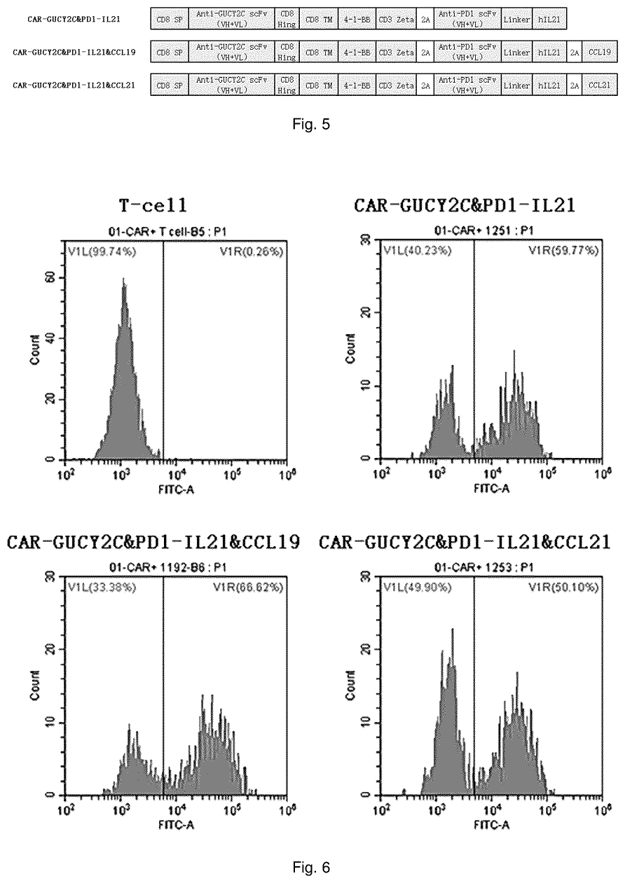

In an optional implementation mode, the structure of the recombinant nucleic acid is shown in CAR-Mesothelin&PD1-IL21&CCL19 or CAR-Mesothelin&PD1-IL21&CCL21 in FIG. 1, or CAR-GUCY2C&PD1-IL21&CCL19 or CAR-GUCY2C&PD1-IL21&CCL21 in FIG. 5. In a third aspect, the present disclosure provides a biomaterial, and the biomaterial comprises any one of the following:
(i) a recombinant vector, herein the recombinant vector comprises the gene according to any one of the aforementioned implementation modes, or the recombinant nucleic acid according to any one of the aforementioned implementation modes; and an original plasmid vector used in the recombinant vector may be routinely selected by those skilled in the art according to actual needs; and
(ii) a construct, herein the construct comprises a non-pathogenic virus, and the non-pathogenic virus comprises the gene, the recombinant nucleic acid, or the recombinant vector in (i) according to any one of the aforementioned implementation modes.

In an optional implementation mode, the non-pathogenic virus comprises a retrovirus, a lentivirus, or an adenovirus.

In a fourth aspect, the present disclosure provides a transgenic immune effector cell, and the transgenic immune effector cell comprises the gene, the recombinant nucleic acid, or the biomaterial according to any one of the aforementioned implementation modes.

In an optional implementation mode, a construction method comprises introducing the gene, the recombinant nucleic acid, or the biomaterial according to any one of the aforementioned implementation modes into an immune effector cell, to obtain the transgenic immune effector cell.

The immune effector cell is selected from at least one of T cell, NK cell, NKT cell, macrophage, or CIK cell.

Preferably, the immune effector cell is the T cell.

In a fifth aspect, the present disclosure provides an application of the gene, the recombinant nucleic acid, the biomaterial, the transgenic immune effector cell, the transgenic immune effector cell constructed by the construction method according to any one of the aforementioned implementation modes in preparation of an anti-tumor product.

Preferably, the tumor comprises a solid tumor.

In a sixth aspect, the present disclosure provides an anti-tumor drug, and the drug comprises at least one of the gene, the recombinant nucleic acid, the biomaterial, or the transgenic immune effector cell according to any one of the aforementioned implementation modes.

Preferably, the tumor comprises a solid tumor.

In a seventh aspect, the present disclosure provides a method for treating a tumor, and the method comprises administering a therapeutically effective amount of at least one of the gene, the recombinant nucleic acid, the biomaterial, or the transgenic immune effector cell according to any one of the aforementioned implementation modes to a subject. Preferably, the tumor comprises a solid tumor.

In an eighth aspect, the present disclosure provides a pharmaceutical composition, and it comprises at least one of the gene, the recombinant nucleic acid, the biomaterial, or the transgenic immune effector cell according to any one of the aforementioned implementation modes, as well as a pharmaceutically acceptable carrier. The pharmaceutically acceptable carrier refers to an inactive substance used in preparations to deliver drugs such as the antibody. The pharmaceutically acceptable carrier may be an anti-adhesive agent, an adhesive, a coating, a disintegrant, a filler, a diluent, a preservative, a sweetener, an absorption retardant, a wetting agent, an emulsifier, a buffering agent and the like. In some implementation modes, the pharmaceutical composition is used to treat the tumor.

Some implementation modes of the present disclosure are described in detail below in combination with the drawings. In the case without conflicting, the following embodiments and the features in the embodiments may be combined with each other.

### Embodiment 1: Construction of Mesothelin-Targeting Lentivirus Expressing Anti PD1-IL-21 Fusion Protein and CCL19

This embodiment constructed a Mesothelin-targeting lentivirus expressing Anti PD1-IL-21 fusion protein and CCL19, and steps were as follows:

### (1) Preparation of Recombinant Virus Vector

A recombinant nucleic acid CAR-Mesothelin&PD1-IL21&CCL19 was synthesized by artificial gene synthesis, as shown in the second Schema Graph of FIG. 1. The nucleotide sequence of the recombinant nucleic acid was shown in SEQ ID NO: 6, a nucleotide fragment was constructed onto a lentiviral vector according to a cleavage site of the lentiviral vector, to design a primer, and the correctness of the vector construction was verified by a sequencing result.

The nucleotide sequence of the recombinant nucleic acid CAR-Mesothelin&PD1-IL21&CCL19 (SEQ ID NO: 6):

(SEQ ID NO: 6)
ATGGCCTTACCAGTGACCGCCTTGCTCCTGCCGCTGGCCTTGCTG
CTCCACGCCGCCAGGCCGCAAGTCCAGCTCCAGCAGTCGGGCCCA
GAGTTGGAGAAGCCTGGGGCGAGCGTGAAGCTTTCATGCAAAGCC
TCAGGCTACTCCTTTACTGGATACACGATGAATTGGGTGAAACAG
TCGCATGGAAAGTCACTGGAATGGATCGGTCTGATTACGCCCTAC
AACGGCGCCTCCAGCTACAACCAGAAGTTCAGGGGAAAGGCGACC
CTTACTGTCGACAAGTCGTCAAGCACCGCCTACATGGACCTCCTG
TCCCTGACCTCCGAAGATAGCGCGGTCTACTTTTGTGCACGCGGA
GGTTACGATGGACGGGGATTCGACTACTGGGGCCAGGGAACCACT
GTCACCGTGTCGAGCGGTGGCGGTGGCTCGGGCGGTGGTGGGTCG
GGTGGCGGCGGATCTGATATCGAACTCACTCAGTCCCCAGCAATC
ATGTCCGCTTCACCGGGAGAAAAGGTGACCATGACTTGCTCGGCC
TCCTCGTCCGTGTCATACATGCACTGGTACCAACAAAAATCGGGG
ACCTCCCCTAAGAGATGGATCTACGATACCAGCAAACTGGCTTCA
GGCGTGCCGGGACGCTTCTCGGGTTCGGGGAGCGGAAATTCGTAT
TCGTTGACCATTTCGTCCGTGGAAGCCGAGGACGACGCAACTTAT
TACTGCCAACAGTGGTCAAAGCACCCGCTCACTTTCGGAGCCGGC
ACTAAGCTGGAGATCAAGACCACGACGCCAGCGCCGCGACCACCA
ACACCGGCGCCCACCATCGCGTCGCAGCCCCTGTCCCTGCGCCCA
GAGGCGTGCCGGCCAGCGGGGGGGGCGCAGTGCACACGAGGGGGC
TGGACTTCGCCTGTGATATCTACATCTGGGCGCCCTTGGCCGGGA
CTTGTGGGGTCCTTCTCCTGTCACTGGTTATCACCCTTTACTGCA
AACGGGGCAGAAAGAAACTCCTGTATATATTCAAACAACCATTTA
TGAGACCAGTACAAACTACTCAAGAGGAAGATGGCTGTAGCTGCC
GATTTCCAGAAGAAGAAGAAGGAGGATGTGAACTGAGAGTGAAGT
TCAGCAGGAGCGCAGACGCCCCCGCGTACAAGCAGGGCCAGAACC
AGCTCTATAACGAGCTCAATCTAGGACGAAGAGAGGAGTACGATG
TTTTGGACAAGAGACGTGGCCGGGACCCTGAGATGGGGGGAAAGC
CGAGAAGGAAGAACCCTCAGGAAGGCCTGTACAATGAACTGCAGA
AAGATAAGATGGCGGAGGCCTACAGTGAGATTGGGATGAAAGGCG
AGCGCCGGAGGGGCAAGGGGCACGATGGCCTTTACCAGGGTCTCA
GTACAGCCACCAAGGACACCTACGACGCCCTTCACATGCAGGCCC
TGCCCCCTCGCGCCACTAACTTCTCCCTGTTGAAACAAGCAGGGG
ATGTCGAAGAGAATCCCGGGCCAATGGGATGGTCTTGTATTATTC
TGTTTCTGGTGGCAACTGCTACTGGGGTGCATAGTGAGATCGTGC
TGACCCAGTCTCCAGCCACACTGAGCCTGTCTCCTGGCGAGAGAG
CCACCCTGTCTTGTAGGGCCAGCCAGTCCGTGAGCTCTTACCTGG
CCTGGTATCAGCAGAAGCCAGGCCAGGCCCCAAGACTGCTGATCT
ACGACGCCTCCAACAGAGCCACCGGCATCCCAGCCAGATTTTCTG
GCTCCGGCTCTGGCACCGACTTCACACTGACCATCAGCTCTCTGG
AGCCAGAGGATTTCGCCGTGTATTACTGCCAGCAGAGCTCTAACT
GGCCAAGAACATTCGGGCAGGGGACCAAGGTGGAAATCAAGAGGG
GGGGGGGGGCTCTGGCGGGGGGGGCTCCGGCGGCGGCGGCTCTCA
GGTGCAGCTGGTGGAGAGCGGCGGCGGAGTGGTGCAGCCAGGCAG
ATCTCTGAGACTGGATTGCAAGGCCAGCGGCATCACCTTCAGCAA
TTCCGGCATGCACTGGGTGCGGCAGGCCCCCGGCAAGGGCCTGGA
GTGGGTGGCCGTGATCTGGTATGACGGCTCTAAGCGGTACTATGC
CGACTCTGTGAAGGGCAGATTCACCATCTCCAGGGACAACTCCAA
GAATACCCTGTTCCTGCAGATGAACAGCCTGAGGGCCGAGGATAC
CGCCGTGTACTATTGCGCCACCAACGACGATTACTGGGGCCAGGG
CACACTGGTGACCGTGTCCAGGGGGGGGGGGGCTCTGGCGGCGGC
GGCTCCGGCGGCGGCGGCTCTATGCACAAATCAAGCTCCCAAGGT
CAAGATCGCCACATGATTAGAATGCGTCAACTTATAGATATTGTT
GATCAGCTGAAAAATTATGTGAATGACTTGGTCCCTGAATTTCTG
CCAGCTCCAGAAGATGTAGAGACAAACTGTGAGTGGTCAGCTTTT
TCCTGTTTTCAGAAGGCCCAACTAAAGTCAGCAAATACAGGAAAC
AATGAAAGGATAATCAATGTATCAATTAAAAAGCTGAAGAGGAAA
CCACCTTCCACAAATGCAGGGAGAAGACAGAAACACAGACTAACA
TGCCCTTCATGTGATTCTTATGAGAAAAAACCACCCAAAGAATTC
CTAGAAAGATTCAAATCACTTCTCCAAAAGATGATTCATCAGCAT
CTGTCCTCTAGAACACACGGAAGTGAAGATTCCGAGGGCAGGGGA
AGTCTTCTAACATGCGGTGACGTGGAGGAAAACCCAGGTCCAATG
GCCCTGCTACTGGCCCTCAGCCTGCTGGTTCTCTGGACTTCCCCA
GCCCCAACTCTGAGTGGCACCAATGATGCTGAAGACTGCTGCCTG
TCTGTGACCCAGAAACCCATCCCTGGGTACATCGTGAGGAACTTC
CACTACCTTCTCATCAAGGATGGCTGCAGGGTGCCTGCTGTAGTG
TTCACCACACTGAGGGGCCGCCAGCTCTGTGCACCCCCAGACCAG
CCCTGGGTAGAACGCATCATCCAGAGACTGCAGAGGACCTCAGCC
AAGATGAAGCGCCGCAGCAGTTAA.

### (2) Packaging and Concentration of Lentivirus

293t cells were inoculated at a density of 8×10⁶ cells/150 mm² in a culture dish, the status of the cells was observed on the next day, the 3rd generation lentivirus packaging vector was co-transfected to the 293t cells with a PEI transfection method, solution was changed after 6 hours of transfection, a DMEM medium comprising 10% fetal bovine serum was added to a 15 mL/150 mm2 culture dish, a virus supernatant was collected after 48 h and 72 h of the transfection, it was centrifuged at 2000 rpm and 4° C. for 10 min, cell debris were removed, and then impurities were filtered with a 0.45 micron filter. A filtered lentivirus suspension was concentrated at 25000 rpm and 4° C. for 2 h, an appropriate amount of a medium was added to the concentrated virus for resuspension, and it was placed at −80° C. for storage.

### Embodiment 2: Construction of Mesothelin-Targeting Lentivirus Expressing Anti PD1-IL-21 Fusion Protein and CCL21

The difference between this embodiment and Embodiment 1 was that CCL19 was replaced with CCL21, as shown in the third Schema Graph of FIG. 1, and the rest parts were consistent with Embodiment 1, a Mesothelin-targeting lentivirus expressing Anti PD1-IL-21 fusion protein and CCL21 was obtained.

### Embodiment 3: Generation of CAR-Mesothelin&PD1-IL21&CCL19 Cell

This embodiment used the lentivirus provided in Embodiment 1 to generate a CART cell, comprising the following steps:

20 mL of blood was drawn, peripheral blood mononuclear cells (PBMC) were separated by Ficall gradient centrifugation. T cells were separated with a T cell sorting kit (product number: 19051) of Stemcell Company, the separated T cells were resuspended to 1×10⁶ cells/mL with 5% human AB serum and 300 units/mL IL-2 X-VIVO 15 medium, and beads were washed with 1% FBS X-VIVO 15. The pre-cleaned magnetic beads (Cat #40203D, 10 mL, Life technology) were added in a volume ratio of magnetic beads: T cells=2:1, after 2˜3 days, the T cells were resuspended to 3˜5×10⁶ cells/mL with a fresh medium, the lentiviruses were added according to a MOI=10 value, and 8 μg/mL of Polybrene was added at the same time. After 4˜6 h, the cells were diluted to 1×10⁶ cells/mL by adding the medium. On the next day, the fresh medium was replaced, so that the cell concentration was maintained at 0.2˜0.3×10⁶ PBMC/mL, and then the medium was changed once every 2˜3 days. The CART cells obtained were named as a CAR-Mesothelin&PD1-IL21&CCL19 group.

### Embodiment 4: Generation of CAR-Mesothelin&PD1-IL21&CCL21 Cell

This embodiment used the lentivirus provided in Embodiment 2 to generate a CART cell according to the method in Embodiment 3, and it was named as a CAR-Mesothelin&PD1-IL21&CCL21 group.

### Embodiment 5: Construction of GUCY2C-Targeting Lentivirus Expressing Anti PD1-IL-21 Fusion Protein and CCL19 or CCL21

This embodiment constructed a GUCY2C-targeting lentivirus expressing Anti PD1-IL-21 fusion protein and CCL19 or CCL19, and steps were as follows:

### (1) Preparation of Recombinant Virus Vector

A recombinant nucleic acid CAR-GUCY2C&PD1-IL21&CCL19 was synthesized by artificial gene synthesis, as shown in the second Schema Graph of FIG. 5, the nucleotide sequence of the recombinant nucleic acid was shown in SEQ ID NO: 7; and a recombinant nucleic acid CAR-GUCY2C&PD1-IL21&CCL21 was synthesized by artificial gene synthesis, as shown in the third Schema Graph of FIG. 5, the nucleotide sequence of the recombinant nucleic acid was shown in SEQ ID NO: 8. A nucleotide fragment was constructed onto a lentiviral vector according to a cleavage site of the lentiviral vector, to design a primer, and the correctness of the vector construction was verified by a sequencing result.

The nucleotide sequence of the recombinant nucleic acid CAR-GUCY2C&PD1-IL21&CCL19 (SEQ ID NO: 7):

ATGGCCTTACCAGTGACCGCCTTGCTCCTGCCGCTGGCCTTGCTG
CTCCACGCCGCCAGGCCGGAGGTCCAGCTGCAGCAGTCTGGACCT
GAGCTGGTGAAGCCTGGGGCTTCAGTGAAGATATCCTGCAAGACT
TCTGGATACACATTCACTGAATACACCATGCACTGGGTGAAGCAG
AGCCATGGAAAGAGCCTTGAGTGGATTGGAGGTATTAATCCTAAC
AATGGTAATACTAACTACAACCAGAAATTCAAGGGCAAGGCCACA
TTGACTGTCGACAAGTCCTCCAGCACAGCCTACATGGAACTCCGC
AGCCTGACATCTGAGGATTCTGCGGTCTATTACTGTGGAAGATCC
CCATTCGTTCATTACTACGACTACTATGCTATGGACTACTGGGGT
CAAGGAACCTCAGTCACCGTCTCCTCAGGAGGAGGAGGCTCCGGC
GGAGGAGGCTCTGGAGGAGGAGGCAGCGACATTGTGCTGACCCAA
TCTCCAGCTTCTTTGGCTGTGTCTCTAGGGCAGAGGGCCACCATC
TCCTGCAGAGCCAGCGAAAGTGTTTATAATTCTGGCATTAGTTTT
ATGAACTGGTTCCAACAGAAACCAGGACAGCCACCCAAACTCCTC
ATCTATGCTGCATCCAACCAAGGATCCGGGGTCCCTGCCAGGTTT
AGTGGCAGTGGGTCTGGGACAGACTTCAGCCTCAACATCCATCCT
ATGGAGGAGGATGATACTGCAATGTATTTCTGTCAGCAAAGTAAG
GAGGTTCCATTCACGTTCGGCTCGGGGACAAACTTGGAAATAAAA
CGGACCACGACGCCAGCGCCGCGACCACCAACACCGGCGCCCACC
ATCGCGTCGCAGCCCCTGTCCCTGCGCCCAGAGGCGTGCCGGCCA
GCGGGGGGGGCGCAGTGCACACGAGGGGGCTGGACTTCGCCTGTG
ATATCTACATCTGGGCGCCCTTGGCCGGGACTTGTGGGGTCCTTC
TCCTGTCACTGGTTATCACCCTTTACTGCAAACGGGGCAGAAAGA
AACTCCTGTATATATTCAAACAACCATTTATGAGACCAGTACAAA
CTACTCAAGAGGAAGATGGCTGTAGCTGCCGATTTCCAGAAGAAG
AAGAAGGAGGATGTGAACTGAGAGTGAAGTTCAGCAGGAGCGCAG
ACGCCCCCGCGTACAAGCAGGGCCAGAACCAGCTCTATAACGAGC
TCAATCTAGGACGAAGAGAGGAGTACGATGTTTTGGACAAGAGAC
GTGGCCGGGACCCTGAGATGGGGGGAAAGCCGAGAAGGAAGAACC
CTCAGGAAGGCCTGTACAATGAACTGCAGAAAGATAAGATGGCGG
AGGCCTACAGTGAGATTGGGATGAAAGGCGAGCGCCGGAGGGGCA
AGGGGCACGATGGCCTTTACCAGGGTCTCAGTACAGCCACCAAGG
ACACCTACGACGCCCTTCACATGCAGGCCCTGCCCCCTCGCGCCA
CTAACTTCTCCCTGTTGAAACAAGCAGGGGATGTCGAAGAGAATC
CCGGGCCAATGGGATGGTCTTGTATTATTCTGTTTCTGGTGGCAA
CTGCTACTGGGGTGCATAGTGAGATCGTGCTGACCCAGTCTCCAG
CCACACTGAGCCTGTCTCCTGGCGAGAGAGCCACCCTGTCTTGTA
GGGCCAGCCAGTCCGTGAGCTCTTACCTGGCCTGGTATCAGCAGA
AGCCAGGCCAGGCCCCAAGACTGCTGATCTACGACGCCTCCAACA
GAGCCACCGGCATCCCAGCCAGATTTTCTGGCTCCGGCTCTGGCA
CCGACTTCACACTGACCATCAGCTCTCTGGAGCCAGAGGATTTCG
CCGTGTATTACTGCCAGCAGAGCTCTAACTGGCCAAGAACATTCG
GGCAGGGGACCAAGGTGGAAATCAAGAGGGGGGGGGGGGCTCTGG
CGGCGGCGGCTCCGGCGGCGGCGGCTCTCAGGTGCAGCTGGTGGA
GAGCGGCGGCGGAGTGGTGCAGCCAGGCAGATCTCTGAGACTGGA
TTGCAAGGCCAGCGGCATCACCTTCAGCAATTCCGGCATGCACTG
GGTGCGGCAGGCCCCCGGCAAGGGCCTGGAGTGGGTGGCCGTGAT
CTGGTATGACGGCTCTAAGCGGTACTATGCCGACTCTGTGAAGGG
CAGATTCACCATCTCCAGGGACAACTCCAAGAATACCCTGTTCCT
GCAGATGAACAGCCTGAGGGCCGAGGATACCGCCGTGTACTATTG
CGCCACCAACGACGATTACTGGGGCCAGGGCACACTGGTGACCGT
GTCCAGCGGCGGCGGCGGCTCTGGCGGCGGCGGCTCCGGGGGGGG
CGGCTCTATGCACAAATCAAGCTCCCAAGGTCAAGATCGCCACAT
GATTAGAATGCGTCAACTTATAGATATTGTTGATCAGCTGAAAAA
TTATGTGAATGACTTGGTCCCTGAATTTCTGCCAGCTCCAGAAGA
TGTAGAGACAAACTGTGAGTGGTCAGCTTTTTCCTGTTTTCAGAA
GGCCCAACTAAAGTCAGCAAATACAGGAAACAATGAAAGGATAAT
CAATGTATCAATTAAAAAGCTGAAGAGGAAACCACCTTCCACAAA
TGCAGGGAGAAGACAGAAACACAGACTAACATGCCCTTCATGTGA
TTCTTATGAGAAAAAACCACCCAAAGAATTCCTAGAAAGATTCAA
ATCACTTCTCCAAAAGATGATTCATCAGCATCTGTCCTCTAGAAC
ACACGGAAGTGAAGATTCCGAGGGCAGGGGAAGTCTTCTAACATG
CGGTGACGTGGAGGAAAACCCAGGTCCAATGGCCCTGCTACTGGC
CCTCAGCCTGCTGGTTCTCTGGACTTCCCCAGCCCCAACTCTGAG
TGGCACCAATGATGCTGAAGACTGCTGCCTGTCTGTGACCCAGAA
ACCCATCCCTGGGTACATCGTGAGGAACTTCCACTACCTTCTCAT
CAAGGATGGCTGCAGGGTGCCTGCTGTAGTGTTCACCACACTGAG
GGGCCGCCAGCTCTGTGCACCCCCAGACCAGCCCTGGGTAGAACG
CATCATCCAGAGACTGCAGAGGACCTCAGCCAAGATGAAGCGCCG
CAGCAGTTAATGA.

The nucleotide sequence of the recombinant nucleic acid CAR-GUCY2C&PD1-IL21&CCL21 (SEQ ID NO: 8):

ATGGCCTTACCAGTGACCGCCTTGCTCCTGCCGCTGGCCTTGCTG
CTCCACGCCGCCAGGCCGGAGGTCCAGCTGCAGCAGTCTGGACCT
GAGCTGGTGAAGCCTGGGGCTTCAGTGAAGATATCCTGCAAGACT
TCTGGATACACATTCACTGAATACACCATGCACTGGGTGAAGCAG
AGCCATGGAAAGAGCCTTGAGTGGATTGGAGGTATTAATCCTAAC
AATGGTAATACTAACTACAACCAGAAATTCAAGGGCAAGGCCACA
TTGACTGTCGACAAGTCCTCCAGCACAGCCTACATGGAACTCCGC
AGCCTGACATCTGAGGATTCTGCGGTCTATTACTGTGGAAGATCC
CCATTCGTTCATTACTACGACTACTATGCTATGGACTACTGGGGT
CAAGGAACCTCAGTCACCGTCTCCTCAGGAGGAGGAGGCTCCGGC
GGAGGAGGCTCTGGAGGAGGAGGCAGCGACATTGTGCTGACCCAA
TCTCCAGCTTCTTTGGCTGTGTCTCTAGGGCAGAGGGCCACCATC
TCCTGCAGAGCCAGCGAAAGTGTTTATAATTCTGGCATTAGTTTT
ATGAACTGGTTCCAACAGAAACCAGGACAGCCACCCAAACTCCTC
ATCTATGCTGCATCCAACCAAGGATCCGGGGTCCCTGCCAGGTTT
AGTGGCAGTGGGTCTGGGACAGACTTCAGCCTCAACATCCATCCT
ATGGAGGAGGATGATACTGCAATGTATTTCTGTCAGCAAAGTAAG
GAGGTTCCATTCACGTTCGGCTCGGGGACAAACTTGGAAATAAAA
CGGACCACGACGCCAGCGCCGCGACCACCAACACCGGCGCCCACC
ATCGCGTCGCAGCCCCTGTCCCTGCGCCCAGAGGCGTGCCGGCCA
GCGGGGGGGGCGCAGTGCACACGAGGGGGCTGGACTTCGCCTGTG
ATATCTACATCTGGGCGCCCTTGGCCGGGACTTGTGGGGTCCTTC
TCCTGTCACTGGTTATCACCCTTTACTGCAAACGGGGCAGAAAGA
AACTCCTGTATATATTCAAACAACCATTTATGAGACCAGTACAAA
CTACTCAAGAGGAAGATGGCTGTAGCTGCCGATTTCCAGAAGAAG
AAGAAGGAGGATGTGAACTGAGAGTGAAGTTCAGCAGGAGCGCAG
ACGCCCCCGCGTACAAGCAGGGCCAGAACCAGCTCTATAACGAGC
TCAATCTAGGACGAAGAGAGGAGTACGATGTTTTGGACAAGAGAC
GTGGCCGGGACCCTGAGATGGGGGGAAAGCCGAGAAGGAAGAACC
CTCAGGAAGGCCTGTACAATGAACTGCAGAAAGATAAGATGGCGG
AGGCCTACAGTGAGATTGGGATGAAAGGCGAGCGCCGGAGGGGCA
AGGGGCACGATGGCCTTTACCAGGGTCTCAGTACAGCCACCAAGG
ACACCTACGACGCCCTTCACATGCAGGCCCTGCCCCCTCGCGCCA
CTAACTTCTCCCTGTTGAAACAAGCAGGGGATGTCGAAGAGAATC
CCGGGCCAATGGGATGGTCTTGTATTATTCTGTTTCTGGTGGCAA
CTGCTACTGGGGTGCATAGTGAGATCGTGCTGACCCAGTCTCCAG
CCACACTGAGCCTGTCTCCTGGCGAGAGAGCCACCCTGTCTTGTA
GGGCCAGCCAGTCCGTGAGCTCTTACCTGGCCTGGTATCAGCAGA
AGCCAGGCCAGGCCCCAAGACTGCTGATCTACGACGCCTCCAACA
GAGCCACCGGCATCCCAGCCAGATTTTCTGGCTCCGGCTCTGGCA
CCGACTTCACACTGACCATCAGCTCTCTGGAGCCAGAGGATTTCG
CCGTGTATTACTGCCAGCAGAGCTCTAACTGGCCAAGAACATTCG
GGCAGGGGACCAAGGTGGAAATCAAGAGGGGGGGGGGGGCTCTGG
CGGCGGCGGCTCCGGCGGCGGCGGCTCTCAGGTGCAGCTGGTGGA
GAGCGGCGGCGGAGTGGTGCAGCCAGGCAGATCTCTGAGACTGGA
TTGCAAGGCCAGCGGCATCACCTTCAGCAATTCCGGCATGCACTG
GGTGCGGCAGGCCCCCGGCAAGGGCCTGGAGTGGGTGGCCGTGAT
CTGGTATGACGGCTCTAAGCGGTACTATGCCGACTCTGTGAAGGG
CAGATTCACCATCTCCAGGGACAACTCCAAGAATACCCTGTTCCT
GCAGATGAACAGCCTGAGGGCCGAGGATACCGCCGTGTACTATTG
CGCCACCAACGACGATTACTGGGGCCAGGGCACACTGGTGACCGT
GTCCAGCGGCGGCGGCGGCTCTGGCGGCGGCGGCTCCGGCGGCGG
CGGCTCTATGCACAAATCAAGCTCCCAAGGTCAAGATCGCCACAT
GATTAGAATGCGTCAACTTATAGATATTGTTGATCAGCTGAAAAA
TTATGTGAATGACTTGGTCCCTGAATTTCTGCCAGCTCCAGAAGA
TGTAGAGACAAACTGTGAGTGGTCAGCTTTTTCCTGTTTTCAGAA
GGCCCAACTAAAGTCAGCAAATACAGGAAACAATGAAAGGATAAT
CAATGTATCAATTAAAAAGCTGAAGAGGAAACCACCTTCCACAAA
TGCAGGGAGAAGACAGAAACACAGACTAACATGCCCTTCATGTGA
TTCTTATGAGAAAAAACCACCCAAAGAATTCCTAGAAAGATTCAA
ATCACTTCTCCAAAAGATGATTCATCAGCATCTGTCCTCTAGAAC
ACACGGAAGTGAAGATTCCGAGGGCAGGGGAAGTCTTCTAACATG
CGGTGACGTGGAGGAAAACCCAGGTCCAATGGCTCAGTCACTGGC
TCTGAGCCTCCTTATCCTGGTTCTGGCCTTTGGCATCCCCAGGAC
CCAAGGCAGTGATGGAGGGGCTCAGGACTGTTGCCTCAAGTACAG
CCAAAGGAAGATTCCCGCCAAGGTTGTCCGCAGCTACCGGAAGCA
GGAACCAAGCTTAGGCTGCTCCATCCCAGCTATCCTGTTCTTGCC
CCGCAAGCGCTCTCAGGCAGAGCTATGTGCAGACCCAAAGGAGCT
CTGGGTGCAGCAGCTGATGCAGCATCTGGACAAGACACCATCCCC
ACAGAAACCAGCCCAGGGCTGCAGGAAGGACAGGGGGGCCTCCAA
GACTGGCAAGAAAGGAAAGGGCTCCAAAGGCTGCAAGAGGACTGA
GCGGTCACAGACCCCTAAAGGGCCATAATGA.

### (2) Packaging and Concentration of Lentivirus

293t cells were inoculated at a density of 8×10⁶ cells/150 mm² in a culture dish, the status of the cells was observed on the next day, the 3rd generation lentivirus packaging vector was co-transfected to the 293t cells with a PEI transfection method, solution was changed after 6 hours of transfection, a DMEM medium comprising 10% fetal bovine serum was added to a 15 mL/150 mm² culture dish, a virus supernatant was collected after 48 h and 72 h of the transfection, it was centrifuged at 2000 rpm and 4° C. for 10 min, cell debris were removed, and then impurities were filtered with a 0.45 micron filter. A filtered lentivirus suspension was concentrated at 25000 rpm and 4° C. for 2 h, an appropriate amount of a medium was added to the concentrated virus for resuspension, and it was placed at −80° C. for storage.

### Embodiment 6: Generation of CART Cell: CAR-GUCY2C&PD1-IL21&CCL19 and CAR-GUCY2C&PD1-IL21&CCL21

CART cells were generated with the GUCY2C-targeting lentivirus expressing Anti PD1-IL-21 fusion protein and CCL19 and the GUCY2C-targeting lentivirus expressing Anti PD1-IL-21 fusion protein and CCL21 constructed in Embodiment 5 respectively according to the method in Embodiment 3, which were named as CAR-GUCY2C&PD1-IL21&CCL19 and CAR-GUCY2C&PD1-IL21&CCL21 respectively.

### Comparative Example 1

This Comparative example constructed a lentivirus, and the difference from Embodiment 1 was that the CCL19 coding region was omitted, and the rest parts were consistent with Embodiment 1, as shown in the first Schema Graph of FIG. 1. Then, this lentivirus was used to generate a CART cell according to the method in Embodiment 3, and it was named as a CAR-Mesothelin&PD1-IL21 group.

### Comparative Example 2

This Comparative example constructed a CART cell: CAR-GUCY2C&PD1-IL21. Firstly, a GUCY2C-targeting lentivirus expressing an Anti PD1-IL-21 fusion protein (the difference between the construction method of the Comparative example 2 and that of the GUCY2C-targeting lentivirus expressing the Anti PD1-IL-21 fusion protein and CCL19 in Embodiment 5 was that the nucleic acid omitted the CCL19 coding region, and the rest parts were consistent with Embodiment 5) was constructed, and the structure of the lentivirus was shown in the first Schema Graph in FIG. 5; then, the lentivirus was used to generate the CART cells according to the method in Embodiment 3, and the CART cells obtained were named as CAR-GUCY2C&PD1-IL21.

### Experimental Example 1

After 72 h of virus infection, a flow cytometry was used to analyze the cell positivity rates of CART cells obtained from the CAR-Mesothelin&PD1-IL21&CCL19 group in Embodiment 3, the CAR-Mesothelin&PD1-IL21&CCL21 in Embodiment 4, and group the CAR-Mesothelin&PD1-IL21 group in Comparative example 1.

As shown in FIG. 2, detection results of the CAR positive rates on Day 6 (D6) after T cell infection showed that the CAR positive rate in the CAR-Mesothelin&PD1-IL21 group was 47.00%, the CAR positive rate in the CAR-Mesothelin&PD1-IL21&CCL19 group was 35.50%, and the CAR positive rate in the CAR-Mesothelin&PD1-IL21&CCL21 group was 29.87%; and from the results, it might be seen that the present it disclosure successfully prepared CAR-Mesothelin&PD1-IL21&CCL19 and CAR-Mesothelin&PD1-IL21&CCL21.

### Experimental Example 2

A flow cytometry was used to detect T, B, and NK cell subsets in the infected cells on D6 in the CAR-Mesothelin&PD1-IL21&CCL19 group of Embodiment 3, the CAR-Mesothelin&PD1-IL21&CCL21 group of Embodiment 4, and the CAR-Mesothelin&PD1-IL21 group of Comparative example 1, as well as CD4, CD8, PD1, and PD-L1 subsets in T cells.

As shown in Table 1, it might be seen that: the expression level of PD1 molecules in the PD1-IL21 expression group was decreased, it was indicated that the PD1-IL21 fusion protein might regulate the expression of the PD1 molecules on the T cell surface, and the proportion of PD1 expression on the T cell surface was greatly reduced.

TABLE 1
Representation results of flow cytometry on three groups of CART cells

| | | | | | B cell | NK cell |
|---|---|---|---|---|---|---|
| | T cell subset | | | | subset | subset |
| Group | CD3+ | CD3+CD4+ | CD3+CD8+ | CD3+PD1+ | CD−CD19+ | CD3−CD56+ |
| T-cell | 99.47% | 56.37% | 34.85% | 3.71% | 0.00% | 0.27% |
| CAR-Mesothelin& | 99.43% | 40.33% | 52.87% | 1.07% | 0.03% | 0.53% |
| PD1-IL21 | | | | | | |
| CAR-Mesothelin& | 99.20% | 47.88% | 45.60% | 0.50% | 0.00% | 0.20% |
| PD1-IL21&CCL19 | | | | | | |
| CAR-Mesothelin& | 99.27% | 49.63% | 43.65% | 0.70% | 0.00% | 0.37% |
| PD1-IL21&CCL21 | | | | | | |

### Experimental Example 3

T cells were used as a control, in vitro killing functions of the infected cells on D6 in the CAR-Mesothelin&PD1-IL21&CCL19 group of Embodiment 3 and the CAR-Mesothelin&PD1-IL21&CCL21 group of Embodiment 4 were evaluated, an xCELLigence real-time cell-mediated cytotoxicity system (Acea Biosciences Inc.) was used to evaluate CART cell-mediated cytotoxicity. 1×10⁴ OVCAR3 cells were cultured in a 150 μL growth medium in each well of E-Plate 16 (Acea Biosciences), and cultured overnight in a 37° C. incubator. Electrical impedance was quantified once every 15 min with RTCA DP Analyzer system, RTCA software version 2.0 (Acea Biosciences Inc.). About 24 h later, 50 μL of CART cells (an E:T ratio was 3:1) or 50 μL of a medium was added as negative, the cell-mediated killing was quantified within the next 24 h, and the electrical impedance was read once every 15 min.

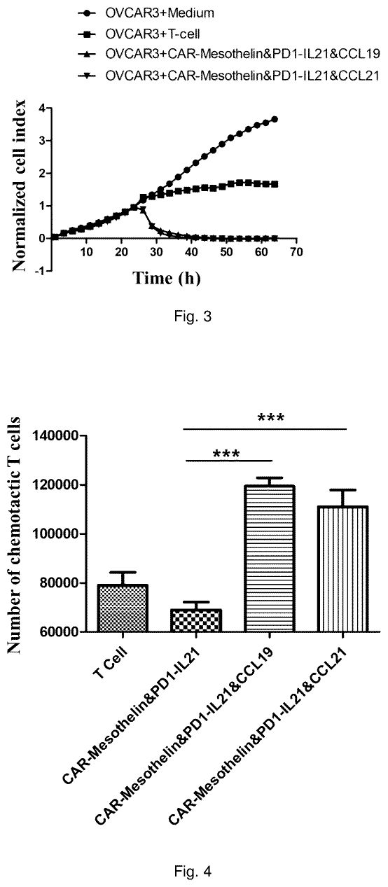

As shown in FIG. 3, it might be seen that when co-cultured with target cells in vitro, compared with the T cells, the cells in the CAR-Mesothelin&PD1-IL21&CCL19 group and CAR-Mesothelin&PD1-IL21&CCL21 group all showed very significant tumor killing ability.

### Experimental Example 4

T cells were used as a control, a chemotaxis experiment was conducted on cells in three groups: the CAR-Mesothelin&PD1-IL21&CCL19 group of Embodiment 3, the CAR-Mesothelin&PD1-IL21&CCL21 group of Embodiment 4, and the CAR-Mesothelin&PD1-IL21 group of Comparative example 1. On the 8th day of culture for the T cells, the cells in the CAR-Mesothelin&PD1-IL21 group, the cells in the CAR-Mesothelin&PD1-IL21&CCL19 group, and the cells in the CAR-Mesothelin&PD1-IL21&CCL21 group, 2 mL, 300 g, and 5 min of a cell culture supernatant was taken from each group, RT centrifugation was performed, and then a supernatant was taken and added to a lower chamber of a transwell plate (Corning, 3422). Three duplication wells were set in each group, and 0.6 mL of the supernatant was added to each well. T-Cells were taken, counted and centrifuged, then resuspend to 2E6/mL with an X-vivo medium, and 100 μL of a cell suspension was added to an upper chamber. After it was placed in an incubator for 6 h of culture, the cell suspension in the lower chamber was collected and counted, and the number of cells migrating to the lower chamber was calculated.

Experimental results were shown in FIG. 4. It might be seen that, compared with the T cells and Comparative example 1, CCL19 and CCL21 secreted in the cell supernatants of the CAR-Mesothelin&PD1-IL21&CCL19 group and the CAR-Mesothelin&PD1-IL21&CCL21 group had a very significant chemotactic effect on the T cells.

### Experimental Example 5

In CAR-GUCY2C&PD1-IL21&CCL19 and CAR-GUCY2C&PD1-IL21&CCL21 generated in Embodiment 6 and CAR-GUCY2C&PD1-IL21 generated in Comparative example 2, the positive rates of CART cells obtained after 72 h of virus infection were analyzed with a flow cytometry.

Experimental results were shown in FIG. 6, and the CAR positive rate detection results of the T cell infection on D6 showed that the CAR positive rate in the CAR-GUCY2C&PD1-IL21 group was 59.77%, the CAR positive rate in the CAR-GUCY2C&PD1-IL21&CCL19 group was 66.62%, and the CAR positive rate in the CAR-GUCY2C&PD1-IL21&CCL21 group was 50.10%; and from the results, it might be seen that: the present disclosure successfully prepared CAR-GUCY2C&PD1-IL21&CCL19 and CAR-GUCY2C&PD1-IL21&CCL21.

### Experimental Example 6

T cells and CART cells CAR-GUCY2C&PD1-IL21 were used as controls, in vitro killing functions of the infected cells on D6 in CAR-GUCY2C&PD1-IL21&CCL19 and CAR-GUCY2C&PD1-IL21&CCL21 of Embodiment 6 were evaluated, an xCELLigence real-time cell-mediated cytotoxicity system (Acea Biosciences Inc.) was used to evaluate CART cell-mediated cytotoxicity. 1×10⁴ OVCAR3 cells were cultured in a 150 μL growth medium in each well of E-Plate 16 (Acea Biosciences), and cultured overnight in a 37° C. incubator. Electrical impedance was quantified once every 15 min with RTCA DP Analyzer system, RTCA software version 2.0 (Acea Biosciences Inc.). About 24 h later, 50 pL of the T cells or 50 μL of CART cells (an E:T ratio was 3:1) or 50 μL of a medium was added as negative, the cell-mediated killing was quantified within the next 24 h, and the electrical impedance was read once every 15 min.

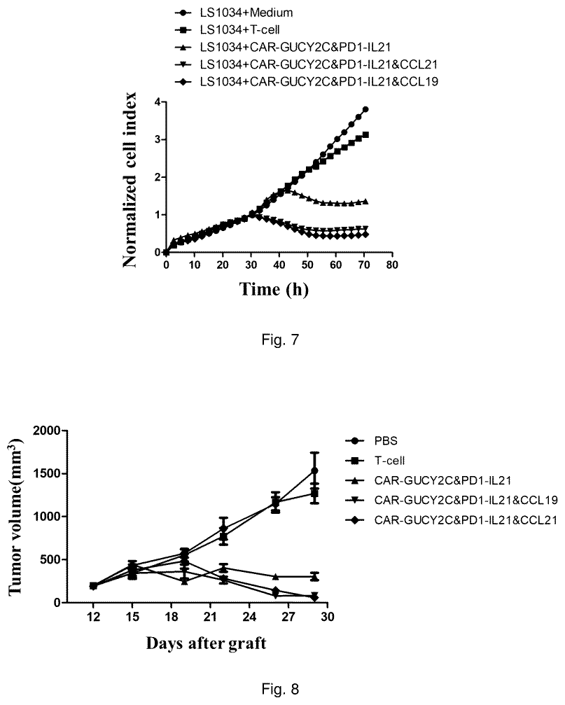

Experimental results were shown in FIG. 7, and the experimental results showed that, when co-cultured with target cells in vitro, compared with the T cells and CAR-GUCY2C&PD1-IL21 cells, the cells in the CAR-GUCY2C&PD1-IL21&CCL19 group and CAR-GUCY2C&PD1-IL21&CCL21 group all had very significant tumor killing ability.

### Experiment Example 7

### Mouse in Vivo Tumor Inhibition Ability Experiment

An LS1034 (human colorectal cancer cell) tumor model was used, 25 NPG mice (Beijing Vitalstar Biotechnology Co., Ltd.) were subcutaneously inoculated, and divided into 5 groups, there were 5 mice in each group. When the tumor grew for 12 days, a tail vein administration mode was used. Each mouse was injected with 5.0E+06 CAR+ cells, T cells, or phosphate buffer (PBS) once, and the tumor volume was measured twice a week. Experimental results were shown in FIG. 8. The results showed that, compared with T cells and CAR-GUCY2C&PD1-IL21 cells, the cells in the CAR-GUCY2C&PD1-IL21&CCL19 group and CAR-GUCY2C&PD1-IL21&CCL21 group had very significant ability to inhibit LS1034 tumor growth in the NPG mice.

From the above embodiments and experimental examples, it may be seen that the fusion of the PD1 antibody and IL-21 in the CART cells is an ideal pathway. PD1 is expressed on the surface of the T cells, and it is CD8+T cells mainly. The PD1 antibody and IL-21 fusion protein formed in this way may selectively bind to the surface of the T cells and CART cells, to exert its dual functions; at the same time, the fusion protein also greatly increases the half-life of the drug due to its increased molecular weight.

The present disclosure constructs CCL19 or CCL21 into the CAR structure by molecular construction, so that the CART cells are allowed to simultaneously express CAR and CCL19 or CCL21 chemokines. When CART kills the tumors, the expressed and secreted CCL19 or CCL21 may recruit and promote the natural T cells to reach the tumor site, to help the CART cells killing the tumors.

Finally, it should be noted that the above embodiments are only used to describe the technical schemes of the present disclosure, and not to limit it; although the present disclosure is described in detail with reference to the aforementioned embodiments, those skilled in the art should understand that: it may still modify the technical schemes recorded in the aforementioned embodiments, or equivalently replace some or all of the technical features therein; and these modifications or replacements do not make the essence of the corresponding technical schemes deviate from the scope of the technical schemes of the various embodiments of the present disclosure.

What is claimed is:

1. A gene comprising three coding regions, wherein
a coding region (I) encodes a chimeric antigen receptor, and the chimeric antigen receptor comprises an extracellular region that specifically recognizes a tumor antigen;
a coding region (II) encodes a fusion protein, and the fusion protein comprises an immune checkpoint antibody and a cytokine; and
a coding region (III) encodes a chemokine.

2. The gene according to claim 1, wherein the tumor antigen is selected from at least one of MSLN, GD2, GPC3, CD19, EGFR VIII, GUCY2C, HER2, MUC16, or Claudin 18.2; and optionally, the tumor antigen is MSLN or GUCY2C.

3. The gene according to claim 2, wherein the extracellular region comprises an anti-MSLN antibody; optionally, the anti-MSLN antibody is a single chain antibody;
optionally,
(a) the amino acid sequence of the anti-MSLN antibody is shown in SEQ ID NO: 1;
(b) the amino acid sequence of the anti-MSLN antibody is a derivative amino acid sequence of SEQ ID NO: 1 that is substituted, deleted, or added with one or more amino acids in the amino acid sequence defined in (a) and encodes a protein with specific recognition of MSLN antigen activity;
(c) the anti-MSLN antibody comprises a heavy chain variable region and a light chain variable region, the heavy chain variable region comprises HCDR1, HCDR2, and HCDR3 in SEQ ID NO: 9, and the light chain variable region comprises LCDR1, LCDR2, and LCDR3 in SEQ ID NO: 10; and optionally, HCDR1, HCDR2, HCDR3, LCDR1, LCDR2, and LCDR3 are defined by Kabat, IMGT, Chothia, Contact, or AbM numbering systems;
(d) the anti-MSLN antibody comprises a heavy chain variable region and a light chain variable region, HCDR1 in the heavy chain variable region comprises an amino acid sequence of SEQ ID NO: 11, HCDR2 comprises an amino acid sequence of SEQ ID NO: 12, and HCDR3 comprises an amino acid sequence of SEQ ID NO: 13; and LCDR1 of the light chain variable region comprises an amino acid sequence of SEQ ID NO: 14, LCDR2 comprises an amino acid sequence of SEQ ID NO: 15, and LCDR3 comprises an amino acid sequence of SEQ ID NO: 16; or
(e) the anti-MSLN antibody comprises a heavy chain variable region and a light chain variable region, wherein the heavy chain variable region comprises an amino acid sequence with at least 85% sequence identity with SEQ ID NO: 9, and the light chain variable region comprises an amino acid sequence with at least 85% sequence identity with SEQ ID NO: 10; optionally, the heavy chain variable region and the light chain variable region comprise HCDR1, HCDR2, HCDR3, LCDR1, LCDR2, and LCDR3 of the anti-MSLN antibody in (c) or (d); and optionally, the anti-MSLN antibody comprises the heavy chain variable region as shown in SEQ ID NO: and the light chain variable region as shown in SEQ ID NO:10.

4. The gene according to claim 2, wherein the chimeric antigen receptor comprises an anti-GUCY2C antibody; optionally, the anti-GUCY2C antibody is a single chain antibody;
optionally,
(a) the anti-GUCY2C antibody comprises a heavy chain variable region and a light chain variable region, the heavy chain variable region comprises HCDR1, HCDR2, and HCDR3 in SEQ ID NO: 17, and the light chain variable region comprises LCDR1, LCDR2, and LCDR3 in SEQ ID NO: 18; optionally, HCDR1, HCDR2, HCDR3, LCDR1, LCDR2, and LCDR3 are defined by Kabat, IMGT, Chothia, Contact, or AbM numbering systems;
(b) the anti-GUCY2C antibody comprises a heavy chain variable region and a light chain variable region, HCDR1 in the heavy chain variable region comprises an amino acid sequence of SEQ ID NO: 20, HCDR2 comprises an amino acid sequence of SEQ ID NO: 21, and HCDR3 comprises an amino acid sequence of SEQ ID NO: 22; LCDR1 in the light chain variable region comprises an amino acid sequence of SEQ ID NO: 23, LCDR2 comprises an amino acid sequence of AAS, and LCDR3 comprises an amino acid sequence of SEQ ID NO: 24;
(c) the anti-GUCY2C antibody comprises a heavy chain variable region and a light chain variable region, wherein the heavy chain variable region comprises an amino acid sequence with at least 85% sequence identity with SEQ ID NO: 17, and the light chain variable region comprises an amino acid sequence with at least 85% sequence identity with SEQ ID NO: 18; optionally, the heavy chain variable region and the light chain variable region comprise HCDR1, HCDR2, HCDR3, LCDR1, LCDR2, and LCDR3 of the anti-GUCY2C antibody in (a) or (b); optionally, the anti-GUCY2C antibody comprises a heavy chain variable region as shown in SEQ ID NO: 17 and a light chain variable region as shown in SEQ ID NO: 18; or
(d) the anti-GUCY2C antibody is a single chain antibody, and it comprises an amino acid sequence with at least 85% sequence identity with SEQ ID NO: 19; optionally, the antibody comprises the heavy chain variable region and the light chain variable region of the anti-GUCY2C antibody in (c); and optionally, the anti-GUCY2C single chain antibody comprises SEQ ID NO: 19.

5. The gene according claims 1, wherein the immune checkpoint is selected from at least one of PD1, PD-L1, TIGIT, LAG3, CTLA4, BTLA or TIM3; and optionally, the immune checkpoint is selected from PD1.

6. The gene according to claim 5, wherein the immune checkpoint antibody is an anti-PD1 antibody; optionally, the anti-PD1 antibody is a single chain antibody;
optionally,
(a) the anti-PD1 antibody comprises a heavy chain variable region and a light chain variable region, the heavy chain variable region comprises HCDR1, HCDR2, and HCDR3 in SEQ ID NO: 25, and the light chain variable region comprises LCDR1, LCDR2, and LCDR3 in SEQ ID NO: 26; optionally, HCDR1, HCDR2, HCDR3, LCDR1, LCDR2, and LCDR3 are defined by Kabat, IMGT, Chothia, Contact, or AbM numbering systems;
(b) the anti-PD1 antibody comprises a heavy chain variable region and a light chain variable region, HCDR1 in the heavy chain variable region comprises an amino acid sequence of SEQ ID NO: 27, HCDR2 comprises an amino acid sequence of SEQ ID NO: 28, and HCDR3 comprises an amino acid sequence of SEQ ID NO: 29; LCDR1 in the light chain variable region comprises an amino acid sequence of SEQ ID NO: 30, LCDR2 comprises an amino acid sequence of SEQ ID NO: 31, and LCDR3 comprises an amino acid sequence of SEQ ID NO: 32;
(c) the amino acid sequence of the anti-PD1 antibody is shown in SEQ ID NO: 2;
(d) the amino acid sequence of the anti-PD1 antibody is a derivative amino acid sequence of SEQ ID NO: 2 that is substituted, deleted, or added with one or more amino acids in the amino acid sequence defined in (c) and encodes a protein with a PD1 targeting function; or
(e) the anti-PD1 antibody comprises a heavy chain variable region and a light chain variable region, wherein the heavy chain variable region comprises an amino acid sequence with at least 85% sequence identity with SEQ ID NO: 25, and the light chain variable region comprises an amino acid sequence with at least 85% sequence identity with SEQ ID NO: 26; optionally, the heavy chain variable region and the light chain variable region comprise HCDR1, HCDR2, HCDR3, LCDR1, LCDR2, and LCDR3 of the anti-PD1 antibody in (a) or (b); and optionally, the anti-PD1 antibody comprises the heavy chain variable region as shown in SEQ ID NO: 25 and the light chain variable region as shown in SEQ ID NO: 26.

7. The gene according to claim 1, wherein the cytokine is (A) or (B);
(A) is selected from at least one of IL-21, IL-23, IL-2, IL-7, IL-9, IL-12, IL-15, or IL-18;
(B) is a protein derived from (A) that has immune cell regulating activity;
optionally, the cytokine is IL-21; and
optionally, IL-21 has an amino acid sequence as shown in SEQ ID NO: 3.

8. The gene according to claim 1, wherein the chemokine is (c) or (d);
(c) is selected from at least one of CXC chemokine, CC chemokine, CX3C chemokine, or XC chemokine;
(d) is a protein derived from (c) that has a function of inducing directed migration of the immune cell;
preferably, the CXC chemokine is selected from at least one of CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 or CXCL17;
preferably, the CC chemokine is selected from at least one of CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27 or CCL28;
preferably, the CX3C chemokine is CX3CL1;
preferably, the XC chemokine is XCL1; and
optionally, the chemokine is CCL19 or CCL21; preferably, CCL19 has an amino acid sequence as shown in SEQ ID NO: 4; and preferably, CCL21 has an amino acid sequence as shown in SEQ ID NO: 5.

9. A recombinant nucleic acid, wherein the recombinant nucleic acid comprises a first nucleic acid molecule, a second nucleic acid molecule, and a third nucleic acid molecule, the first nucleic acid molecule comprises the coding region (I) according to claim 1, the second nucleic acid molecule comprises the coding region (II) according to claim 1, and the third nucleic acid molecule comprises the coding region (III) according to claim 1.

10. The recombinant nucleic acid according to claim 9, wherein the first nucleic acid molecule, the second nucleic acid molecule, and the third nucleic acid molecule are linked by a nucleic acid sequence of a 2A peptide; preferably, the 2A peptide is selected from at least one of P2A, T2A, F2A, or E2A; and
optionally, the first nucleic acid molecule is linked to the second nucleic acid molecule by the nucleic acid sequence of the 2A peptide, and the second nucleic acid molecule is linked to the third nucleic acid molecule by the nucleic acid sequence of the 2A peptide.

11. A biomaterial, wherein the biomaterial comprises (i) or (ii):
(i) a recombinant vector, wherein the recombinant vector comprises the gene according to claim 1, or a recombinant nucleic acid, the recombinant nucleic acid comprises a first nucleic acid molecule, a second nucleic acid molecule, and a third nucleic acid molecule, the first nucleic acid molecule comprises the coding region (I) according to claim 1, the second nucleic acid molecule comprises the coding region (II) according to claim 1, and the third nucleic acid molecule comprises the coding region (III) according to claim 1;
(ii) a construct, wherein the construct comprises a non-pathogenic virus, and the non-pathogenic virus comprises the gene according to claim 1, a recombinant nucleic acid, or the recombinant vector in (i),
the recombinant nucleic acid comprises a first nucleic acid molecule, a second nucleic acid molecule, and a third nucleic acid molecule, the first nucleic acid molecule comprises the coding region (I) according to claim 1, the second nucleic acid molecule comprises the coding region (II) according to claim 1, and the third nucleic acid molecule comprises the coding region (III) according to claim 1;
preferably, the first nucleic acid molecule, the second nucleic acid molecule, and the third nucleic acid molecule are linked by a nucleic acid sequence of a 2A peptide; preferably, the 2A peptide is selected from at least one of P2A, T2A, F2A, or E2A;
more preferably, the first nucleic acid molecule is linked to the second nucleic acid molecule by the nucleic acid sequence of the 2A peptide, and the second nucleic acid molecule is linked to the third nucleic acid molecule by the nucleic acid sequence of the 2A peptide;
optionally, the non-pathogenic virus comprises a retrovirus, a lentivirus, or an adenovirus.

12. A transgenic immune effector cell, wherein the transgenic immune effector cell comprises the gene according to claim 1, a recombinant nucleic acid, or a biomaterial;
wherein the recombinant nucleic acid comprises a first nucleic acid molecule, a second nucleic acid molecule, and a third nucleic acid molecule, the first nucleic acid molecule comprises the coding region (I) according to claim 1, the second nucleic acid molecule comprises the coding region (II) according to claim 1, and the third nucleic acid molecule comprises the coding region (III) according to claim 1;
wherein the biomaterial comprises (i) or (ii):
(i) a recombinant vector, wherein the recombinant vector comprises the gene or the recombinant nucleic acid;
(ii) a construct, wherein the construct comprises a non-pathogenic virus, and the non-pathogenic virus comprises the gene, the recombinant nucleic acid, or the recombinant vector in (i);
preferably, the first nucleic acid molecule, the second nucleic acid molecule, and the third nucleic acid molecule are linked by a nucleic acid sequence of a 2A peptide; preferably, the 2A peptide is selected from at least one of P2A, T2A, F2A, or E2A;
more preferably, the first nucleic acid molecule is linked to the second nucleic acid molecule by the nucleic acid sequence of the 2A peptide, and the second nucleic acid molecule is linked to the third nucleic acid molecule by the nucleic acid sequence of the 2A peptide:
optionally, the immune effector cell is selected from at least one of T cell, NK cell, NKT cell, macrophage, or CIK cell; and
optionally, the immune effector cell is the T cell.

13. The transgenic immune effector cell according to claim 12, wherein the transgenic immune effector cell comprises a transmembrane region and/or an intracellular region;
optionally, the transmembrane region comprises a transmembrane segment of CD8;
optionally, the intracellular region comprises an intracellular segment of an immune co-stimulatory molecule and/or a CD3 Zeta chain;
optionally, the immune co-stimulatory molecule is selected from any one or more of 4-1BB, CD28, CD3, OX-40, CD40L, CD27, CD30, or their derivatives; and optionally, the immune co-stimulatory molecule is selected from 4-1BB.

14. A construction method for the transgenic immune effector cell according to claim 12, wherein the construction method comprises introducing the gene, the recombinant nucleic acid, or the biomaterial into an immune effector cell, to obtain the transgenic immune effector cell;
optionally, the immune effector cell is selected from at least one of T cell, NK cell, NKT cell, macrophage, or CIK cell; and
optionally, the immune effector cell is the T cell;
optionally, the transgenic immune effector cell comprises a transmembrane region and/or an intracellular region;
optionally, the transmembrane region comprises a transmembrane segment of CD8;
optionally, the intracellular region comprises an intracellular segment of an immune co-stimulatory molecule and/or a CD3 Zeta chain;
optionally, the immune co-stimulatory molecule is selected from any one or more of 4-1BB, CD28, CD3, OX-40, CD40L, CD27, CD30, or their derivatives; and optionally, the immune co-stimulatory molecule is selected from 4-1BB.

15. A pharmaceutical composition, wherein it comprises: the gene according to claim 1, a recombinant nucleic acid, a biomaterial, a transgenic immune effector cell or a transgenic immune effector cell constructed by a construction method, and a pharmaceutically acceptable carrier;
wherein the recombinant nucleic acid comprises a first nucleic acid molecule, a second nucleic acid molecule, and a third nucleic acid molecule, the first nucleic acid molecule comprises the coding region (I) according to claim 1, the second nucleic acid molecule comprises the coding region (II) according to claim 1, and the third nucleic acid molecule comprises the coding region (III) according to claim 1;
preferably, the first nucleic acid molecule, the second nucleic acid molecule, and the third nucleic acid molecule are linked by a nucleic acid sequence of a 2A peptide; preferably, the 2A peptide is selected from at least one of P2A, T2A, F2A, or E2A;
more preferably, the first nucleic acid molecule is linked to the second nucleic acid molecule by the nucleic acid sequence of the 2A peptide, and the second nucleic acid molecule is linked to the third nucleic acid molecule by the nucleic acid sequence of the 2A peptide;
the biomaterial comprises (i) or (ii): (i) a recombinant vector, wherein the recombinant vector comprises the gene or the recombinant nucleic acid; (ii) a construct, wherein the construct comprises a non-pathogenic virus, and the non-pathogenic virus comprises the gene, the recombinant nucleic acid, or the recombinant vector in (i);
the transgenic immune effector cell comprises the gene, the recombinant nucleic acid, or the biomaterial;
optionally, the immune effector cell is selected from at least one of T cell, NK cell, NKT cell, macrophage, or CIK cell; and
optionally, the immune effector cell is the T cell;
optionally, the transgenic immune effector cell comprises a transmembrane region and/or an intracellular region;
optionally, the transmembrane region comprises a transmembrane segment of CD8;
optionally, the intracellular region comprises an intracellular segment of an immune co-stimulatory molecule and/or a CD3 Zeta chain;
optionally, the immune co-stimulatory molecule is selected from any one or more of 4-1BB, CD28, CD3, OX-40, CD40L, CD27, CD30, or their derivatives; and optionally, the immune co-stimulatory molecule is selected from 4-1BB;
the construction method comprises introducing the gene, the recombinant nucleic acid, or the biomaterial into an immune effector cell, to obtain the transgenic immune effector cell;
optionally, the immune effector cell is selected from at least one of T cell, NK cell, NKT cell, macrophage, or CIK cell; and
optionally, the immune effector cell is the T cell.

16. An application of the gene according to claim 1, a recombinant nucleic acid, a biomaterial, a transgenic immune effector cell, a transgenic immune effector cell constructed by a construction method, or a pharmaceutical composition in preparation of an anti-tumor product;
wherein the recombinant nucleic acid comprises a first nucleic acid molecule, a second nucleic acid molecule, and a third nucleic acid molecule, the first nucleic acid molecule comprises the coding region (I) according to claim 1, the second nucleic acid molecule comprises the coding region (II) according to claim 1, and the third nucleic acid molecule comprises the coding region (III) according to claim 1;
preferably, the first nucleic acid molecule, the second nucleic acid molecule, and the third nucleic acid molecule are linked by a nucleic acid sequence of a 2A peptide; preferably, the 2A peptide is selected from at least one of P2A, T2A, F2A, or E2A;
more preferably, the first nucleic acid molecule is linked to the second nucleic acid molecule by the nucleic acid sequence of the 2A peptide, and the second nucleic acid molecule is linked to the third nucleic acid molecule by the nucleic acid sequence of the 2A peptide;
the biomaterial comprises (i) or (ii): (i) a recombinant vector, wherein the recombinant vector comprises the gene or the recombinant nucleic acid; (ii) a construct, wherein the construct comprises a non-pathogenic virus, and the non-pathogenic virus comprises the gene, the recombinant nucleic acid, or the recombinant vector in (i);
the transgenic immune effector cell comprises the gene, the recombinant nucleic acid, or the biomaterial;
optionally, the immune effector cell is selected from at least one of T cell, NK cell, NKT cell, macrophage, or CIK cell; and
optionally, the immune effector cell is the T cell;
optionally, the transgenic immune effector cell comprises a transmembrane region and/or an intracellular region;
optionally, the transmembrane region comprises a transmembrane segment of CD8;
optionally, the intracellular region comprises an intracellular segment of an immune co-stimulatory molecule and/or a CD3 Zeta chain;
optionally, the immune co-stimulatory molecule is selected from any one or more of 4-1BB, CD28, CD3. OX-40, CD40L, CD27, CD30, or their derivatives; and optionally, the immune co-stimulatory molecule is selected from 4-1BB;
the construction method comprises introducing the gene, the recombinant nucleic acid, or the biomaterial into an immune effector cell, to obtain the transgenic immune effector cell;
optionally, the immune effector cell is selected from at least one of T cell, NK cell, NKT cell, macrophage, or CIK cell; and
optionally, the immune effector cell is the T cell;
the pharmaceutical composition comprises the gene, the recombinant nucleic acid, the biomaterial, the transgenic immune effector cell or the transgenic immune effector cell constructed by the construction method, and a pharmaceutically acceptable carrier;
and optionally, the tumor comprises a solid tumor.

17. A method for treating a tumor, wherein the method comprises administering a therapeutically effective amount of at least one of the followings to a subject suffering from the tumor:
the gene according to claim 1, a recombinant nucleic acid, a biomaterial, a transgenic immune effector cell, a transgenic immune effector cell constructed by a construction method, or a pharmaceutical composition;
wherein the recombinant nucleic acid comprises a first nucleic acid molecule, a second nucleic acid molecule, and a third nucleic acid molecule, the first nucleic acid molecule comprises the coding region (I) according to claim 1, the second nucleic acid molecule comprises the coding region (II) according to claim 1, and the third nucleic acid molecule comprises the coding region (III) according to claim 1;
preferably, the first nucleic acid molecule, the second nucleic acid molecule, and the third nucleic acid molecule are linked by a nucleic acid sequence of a 2A peptide; preferably, the 2A peptide is selected from at least one of P2A, T2A, F2A, or E2A;
more preferably, the first nucleic acid molecule is linked to the second nucleic acid molecule by the nucleic acid sequence of the 2A peptide, and the second nucleic acid molecule is linked to the third nucleic acid molecule by the nucleic acid sequence of the 2A peptide;
the biomaterial comprises (i) or (ii): (i) a recombinant vector, wherein the recombinant vector comprises the gene or the recombinant nucleic acid; (ii) a construct, wherein the construct comprises a non-pathogenic virus, and the non-pathogenic virus comprises the gene, the recombinant nucleic acid, or the recombinant vector in (i);
the transgenic immune effector cell comprises the gene, the recombinant nucleic acid, or the biomaterial;
optionally, the immune effector cell is selected from at least one of T cell, NK cell, NKT cell, macrophage, or CIK cell; and
optionally, the immune effector cell is the T cell;
optionally, the transgenic immune effector cell comprises a transmembrane region and/or an intracellular region;
optionally, the transmembrane region comprises a transmembrane segment of CD8;
optionally, the intracellular region comprises an intracellular segment of an immune co-stimulatory molecule and/or a CD3 Zeta chain;
optionally, the immune co-stimulatory molecule is selected from any one or more of 4-1BB, CD28, CD3, OX-40, CD40L, CD27, CD30, or their derivatives; and optionally, the immune co-stimulatory molecule is selected from 4-1BB;
the construction method comprises introducing the gene, the recombinant nucleic acid, or the biomaterial into an immune effector cell, to obtain the transgenic immune effector cell;
optionally, the immune effector cell is selected from at least one of T cell, NK cell, NKT cell, macrophage, or CIK cell; and
optionally, the immune effector cell is the T cell;
the pharmaceutical composition comprises the gene, the recombinant nucleic acid, the biomaterial, the transgenic immune effector cell or the transgenic immune effector cell constructed by the construction method, and a pharmaceutically acceptable carrier;
optionally, the tumor comprises a solid tumor.

18. The gene according to claim 1, a recombinant nucleic acid, a biomaterial, a transgenic immune effector cell, a transgenic immune effector cell constructed by a construction method, or a pharmaceutical composition for use in a method for treating a tumor;
wherein the recombinant nucleic acid comprises a first nucleic acid molecule, a second nucleic acid molecule, and a third nucleic acid molecule, the first nucleic acid molecule comprises the coding region (I) according to claim 1, the second nucleic acid molecule comprises the coding region (II) according to claim 1, and the third nucleic acid molecule comprises the coding region (III) according to claim 1;
preferably, the first nucleic acid molecule, the second nucleic acid molecule, and the third nucleic acid molecule are linked by a nucleic acid sequence of a 2A peptide; preferably, the 2A peptide is selected from at least one of P2A, T2A, F2A, or E2A;
more preferably, the first nucleic acid molecule is linked to the second nucleic acid molecule by the nucleic acid sequence of the 2A peptide, and the second nucleic acid molecule is linked to the third nucleic acid molecule by the nucleic acid sequence of the 2A peptide;
the biomaterial comprises (i) or (ii): (i) a recombinant vector, wherein the recombinant vector comprises the gene or the recombinant nucleic acid; (ii) a construct, wherein the construct comprises a non-pathogenic virus, and the non-pathogenic virus comprises the gene, the recombinant nucleic acid, or the recombinant vector in (i);
the transgenic immune effector cell comprises the gene, the recombinant nucleic acid, or the biomaterial;
optionally, the immune effector cell is selected from at least one of T cell, NK cell, NKT cell, macrophage, or CIK cell; and
optionally, the immune effector cell is the T cell;
optionally, the transgenic immune effector cell comprises a transmembrane region and/or
an intracellular region;
optionally, the transmembrane region comprises a transmembrane segment of CD8;
optionally, the intracellular region comprises an intracellular segment of an immune co-stimulatory molecule and/or a CD3 Zeta chain;
optionally, the immune co-stimulatory molecule is selected from any one or more of 4-1BB, CD28, CD3, OX-40, CD40L, CD27, CD30, or their derivatives; and optionally, the immune co-stimulatory molecule is selected from 4-1BB;
the construction method comprises introducing the gene, the recombinant nucleic acid, or the biomaterial into an immune effector cell, to obtain the transgenic immune effector cell;
optionally, the immune effector cell is selected from at least one of T cell, NK cell, NKT cell, macrophage, or CIK cell; and
optionally, the immune effector cell is the T cell;
the pharmaceutical composition comprises the gene, the recombinant nucleic acid, the biomaterial, the transgenic immune effector cell or the transgenic immune effector cell constructed by the construction method, and a pharmaceutically acceptable carrier;
and optionally, the tumor comprises a solid tumor.


SEQUENCE LISTING (32 sequences)

SEQ ID NO: 1 - protein, 240 aa, synthetic construct
QVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIK

SEQ ID NO: 2 - protein, 255 aa, synthetic construct
MGWSCIILFLVATATGVHSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 3 - protein, 139 aa, synthetic construct
MHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 4 - protein, 98 aa, synthetic construct
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 5 - protein, 134 aa, synthetic construct
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 6 - DNA, 3087 bp, synthetic construct
  source 1..3087: mol_type = other DNA
atggccttaccagtgaccgccttgctcctgccgctggccttgctgctccacgccgccaggccgcaagtccagctccagcagtcgggcccagagttggagaagcctggggcgagcgtgaagctttcatgcaaagcctcaggctactcctttactggatacacgatgaattgggtgaaacagtcgcatggaaagtcactggaatggatcggtctgattacgccctacaacggcgcctccagctacaaccagaagttcaggggaaaggcgacccttactgtcgacaagtcgtcaagcaccgcctacatggacctcctgtccctgacctccgaagatagcgcggtctacttttgtgcacgcggaggttacgatggacggggattcgactactggggccagggaaccactgtcaccgtgtcgagcggtggcggtggctcgggcggtggtgggtcgggtggcggcggatctgatatcgaactcactcagtccccagcaatcatgtccgcttcaccgggagaaaaggtgaccatgacttgctcggcctcctcgtccgtgtcatacatgcactggtaccaacaaaaatcggggacctcccctaagagatggatctacgataccagcaaactggcttcaggcgtgccgggacgcttctcgggttcggggagcggaaattcgtattcgttgaccatttcgtccgtggaagccgaggacgacgcaacttattactgccaacagtggtcaaagcacccgctcactttcggagccggcactaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccactaacttctccctgttgaaacaagcaggggatgtcgaagagaatcccgggccaatgggatggtcttgtattattctgtttctggtggcaactgctactggggtgcatagtgagatcgtgctgacccagtctccagccacactgagcctgtctcctggcgagagagccaccctgtcttgtagggccagccagtccgtgagctcttacctggcctggtatcagcagaagccaggccaggccccaagactgctgatctacgacgcctccaacagagccaccggcatcccagccagattttctggctccggctctggcaccgacttcacactgaccatcagctctctggagccagaggatttcgccgtgtattactgccagcagagctctaactggccaagaacattcgggcaggggaccaaggtggaaatcaagaggggcggcggcggctctggcggcggcggctccggcggcggcggctctcaggtgcagctggtggagagcggcggcggagtggtgcagccaggcagatctctgagactggattgcaaggccagcggcatcaccttcagcaattccggcatgcactgggtgcggcaggcccccggcaagggcctggagtgggtggccgtgatctggtatgacggctctaagcggtactatgccgactctgtgaagggcagattcaccatctccagggacaactccaagaataccctgttcctgcagatgaacagcctgagggccgaggataccgccgtgtactattgcgccaccaacgacgattactggggccagggcacactggtgaccgtgtccagcggcggcggcggctctggcggcggcggctccggcggcggcggctctatgcacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccgagggcaggggaagtcttctaacatgcggtgacgtggaggaaaacccaggtccaatggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaa

SEQ ID NO: 7 - DNA, 3120 bp, synthetic construct
  source 1..3120: mol_type = other DNA
atggccttaccagtgaccgccttgctcctgccgctggccttgctgctccacgccgccaggccggaggtccagctgcagcagtctggacctgagctggtgaagcctggggcttcagtgaagatatcctgcaagacttctggatacacattcactgaatacaccatgcactgggtgaagcagagccatggaaagagccttgagtggattggaggtattaatcctaacaatggtaatactaactacaaccagaaattcaagggcaaggccacattgactgtcgacaagtcctccagcacagcctacatggaactccgcagcctgacatctgaggattctgcggtctattactgtggaagatccccattcgttcattactacgactactatgctatggactactggggtcaaggaacctcagtcaccgtctcctcaggaggaggaggctccggcggaggaggctctggaggaggaggcagcgacattgtgctgacccaatctccagcttctttggctgtgtctctagggcagagggccaccatctcctgcagagccagcgaaagtgtttataattctggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccattcacgttcggctcggggacaaacttggaaataaaacggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccactaacttctccctgttgaaacaagcaggggatgtcgaagagaatcccgggccaatgggatggtcttgtattattctgtttctggtggcaactgctactggggtgcatagtgagatcgtgctgacccagtctccagccacactgagcctgtctcctggcgagagagccaccctgtcttgtagggccagccagtccgtgagctcttacctggcctggtatcagcagaagccaggccaggccccaagactgctgatctacgacgcctccaacagagccaccggcatcccagccagattttctggctccggctctggcaccgacttcacactgaccatcagctctctggagccagaggatttcgccgtgtattactgccagcagagctctaactggccaagaacattcgggcaggggaccaaggtggaaatcaagaggggcggcggcggctctggcggcggcggctccggcggcggcggctctcaggtgcagctggtggagagcggcggcggagtggtgcagccaggcagatctctgagactggattgcaaggccagcggcatcaccttcagcaattccggcatgcactgggtgcggcaggcccccggcaagggcctggagtgggtggccgtgatctggtatgacggctctaagcggtactatgccgactctgtgaagggcagattcaccatctccagggacaactccaagaataccctgttcctgcagatgaacagcctgagggccgaggataccgccgtgtactattgcgccaccaacgacgattactggggccagggcacactggtgaccgtgtccagcggcggcggcggctctggcggcggcggctccggcggcggcggctctatgcacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccgagggcaggggaagtcttctaacatgcggtgacgtggaggaaaacccaggtccaatggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaatga

SEQ ID NO: 8 - DNA, 3228 bp, synthetic construct
  source 1..3228: mol_type = other DNA
atggccttaccagtgaccgccttgctcctgccgctggccttgctgctccacgccgccaggccggaggtccagctgcagcagtctggacctgagctggtgaagcctggggcttcagtgaagatatcctgcaagacttctggatacacattcactgaatacaccatgcactgggtgaagcagagccatggaaagagccttgagtggattggaggtattaatcctaacaatggtaatactaactacaaccagaaattcaagggcaaggccacattgactgtcgacaagtcctccagcacagcctacatggaactccgcagcctgacatctgaggattctgcggtctattactgtggaagatccccattcgttcattactacgactactatgctatggactactggggtcaaggaacctcagtcaccgtctcctcaggaggaggaggctccggcggaggaggctctggaggaggaggcagcgacattgtgctgacccaatctccagcttctttggctgtgtctctagggcagagggccaccatctcctgcagagccagcgaaagtgtttataattctggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccattcacgttcggctcggggacaaacttggaaataaaacggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccactaacttctccctgttgaaacaagcaggggatgtcgaagagaatcccgggccaatgggatggtcttgtattattctgtttctggtggcaactgctactggggtgcatagtgagatcgtgctgacccagtctccagccacactgagcctgtctcctggcgagagagccaccctgtcttgtagggccagccagtccgtgagctcttacctggcctggtatcagcagaagccaggccaggccccaagactgctgatctacgacgcctccaacagagccaccggcatcccagccagattttctggctccggctctggcaccgacttcacactgaccatcagctctctggagccagaggatttcgccgtgtattactgccagcagagctctaactggccaagaacattcgggcaggggaccaaggtggaaatcaagaggggcggcggcggctctggcggcggcggctccggcggcggcggctctcaggtgcagctggtggagagcggcggcggagtggtgcagccaggcagatctctgagactggattgcaaggccagcggcatcaccttcagcaattccggcatgcactgggtgcggcaggcccccggcaagggcctggagtgggtggccgtgatctggtatgacggctctaagcggtactatgccgactctgtgaagggcagattcaccatctccagggacaactccaagaataccctgttcctgcagatgaacagcctgagggccgaggataccgccgtgtactattgcgccaccaacgacgattactggggccagggcacactggtgaccgtgtccagcggcggcggcggctctggcggcggcggctccggcggcggcggctctatgcacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccgagggcaggggaagtcttctaacatgcggtgacgtggaggaaaacccaggtccaatggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccataatga

SEQ ID NO: 9 - protein, 119 aa, synthetic construct
QVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 10 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIK

SEQ ID NO: 11 - protein, 5 aa, synthetic construct
GYTMN

SEQ ID NO: 12 - protein, 17 aa, synthetic construct
LITPYNGASSYNQKFRG

SEQ ID NO: 13 - protein, 10 aa, synthetic construct
GGYDGRGFDY

SEQ ID NO: 14 - protein, 10 aa, synthetic construct
SASSSVSYMH

SEQ ID NO: 15 - protein, 7 aa, synthetic construct
DTSKLAS

SEQ ID NO: 16 - protein, 9 aa, synthetic construct
QQWSKHPLT

SEQ ID NO: 17 - protein, 123 aa, synthetic construct
EVQLQQSGPELVKPGASVKISCKTSGYTFTEYTMHWVKQSHGKSLEWIGGINPNNGNTNYNQKFKGKATLTVDKSSSTAYMELRSLTSEDSAVYYCGRSPFVHYYDYYAMDYWGQGTSVTVSS

SEQ ID NO: 18 - protein, 111 aa, synthetic construct
DIVLTQSPASLAVSLGQRATISCRASESVYNSGISFMNWFQQKPGQPPKLLIYAASNQGSGVPARFSGSGSGTDFSLNIHPMEEDDTAMYFCQQSKEVPFTFGSGTNLEIK

SEQ ID NO: 19 - protein, 249 aa, synthetic construct
EVQLQQSGPELVKPGASVKISCKTSGYTFTEYTMHWVKQSHGKSLEWIGGINPNNGNTNYNQKFKGKATLTVDKSSSTAYMELRSLTSEDSAVYYCGRSPFVHYYDYYAMDYWGQGTSVTVSSGGGGSGGGGSGGGGSDIVLTQSPASLAVSLGQRATISCRASESVYNSGISFMNWFQQKPGQPPKLLIYAASNQGSGVPARFSGSGSGTDFSLNIHPMEEDDTAMYFCQQSKEVPFTFGSGTNLEIK

SEQ ID NO: 20 - protein, 8 aa, synthetic construct
GYTFTEYT

SEQ ID NO: 21 - protein, 8 aa, synthetic construct
INPNNGGA

SEQ ID NO: 22 - protein, 16 aa, synthetic construct
ARAPYYYGSSYYAMDY

SEQ ID NO: 23 - protein, 10 aa, synthetic construct
ESVDNYGISF

SEQ ID NO: 24 - protein, 9 aa, synthetic construct
QQSKEVPFT

SEQ ID NO: 25 - protein, 113 aa, synthetic construct [sequence omitted]

SEQ ID NO: 26 - protein, 126 aa, synthetic construct
MGWSCIILFLVATATGVHSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIK

SEQ ID NO: 27 - protein, 5 aa, synthetic construct
NSGMH

SEQ ID NO: 28 - protein, 17 aa, synthetic construct
VIWYDGSKRYYADSVKG

SEQ ID NO: 29 - protein, 4 aa, synthetic construct
NDDY

SEQ ID NO: 30 - protein, 11 aa, synthetic construct
RASQSVSSYLA

SEQ ID NO: 31 - protein, 7 aa, synthetic construct
DASNRAT

SEQ ID NO: 32 - protein, 9 aa, synthetic construct
QQSSNWPRT